(19) United States
(12) Patent Application Publication
(10) Pub. No.: US 2005/0171207 A1
(43) Pub. Date: Aug. 4, 2005

(54) Method and composition for combination treatment of neurodegenerative disorders

(75) Inventors: Adrian Hobden, Salt Lake City, UT (US); Jean-Marc Roch, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/092,118

(22) Filed: Mar. 28, 2005

(30) Foreign Application Priority Data
Sep. 26, 2003 (WO) PCT/US03/30284

(51) Int. Cl. A61K 31/192; A61K 31/194

(52) U.S. Cl. 514/570; 514/571

(57) ABSTRACT

The invention provides compositions and methods for treating neurodegenerative disorders. The method of the invention involves administering to an individual in need of treatment a composition having an R—NSAID and an HMG-CoA reductase inhibitor. The methods and compositions of the invention are useful for treating and preventing neurodegenerative disorders like Alzheimer's disease, dementia, mild cognitive impairment.

### CROSS-REFERENCE TO RELATED APPLICATIONS

This application is a continuation-in-part of PCT/US2003/030284 filed on Sep. 26, 2003, which claims priority to U.S. Provisional Application Ser. No. 60/414,045, filed on Sep. 26, 2002, the contents of both of which are incorporated herein by reference in their entirety.

### FIELD OF THE INVENTION

The invention relates to compositions and methods for the therapeutic treatment of neurodegenerative disorders, and particularly to a composition having an R—NSAID and an HMG-CoA-reductase inhibitor and methods of use thereof.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60 with the risk increasing with age. Younger people can also get AD, but it is much less common. It is estimated that 3 percent of men and women ages 65 to 74 have AD. Almost half of those ages 85 and older may have the disease. AD is not a normal part of aging. Alzheimer's disease is a complex disease that can be caused by genetic and environmental factors.

In 1906, Dr. Alois Alzheimer, noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have also found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause in decline of cognitive function. The number of tangles is more highly correlated with the cognitive decline than amyloid load patients with AD (Albert PNAS 93:13547-13551 (1996)). The cellular, biochemical, and molecular events responsible for neuronal and synaptic loss in AD are not known. A number of studies have demonstrated that amyloid can be directly toxic to neurons (Iversen et al. Biochem. J. 311:1-16 (1995); Weiss et al. J. Neurochem. 62:372-375 (1994); Lorenzo et al. Ann N Y Acad. Sci. 777:89-95 (1996); Storey et al. Neuropathol. Appl. Neurobiol. 2:81-97 (1999), resulting in behavioral impairment (Ma et al. 1996). The toxicity of amyloid or tangles is potentially aggravated by activation of the complement cascade (Rogers et al. PNAS 21:10016-10020 (1992); Rozemuller et al. Res. Immunol. 6:646-9 (1992); Rogers et al. Res Immunol. 6:624-30 (1992); Webster et al. J. Neurochem. 69(1):388-98 (1997)). This suggests involvement of an inflammatory process in AD and neuronal death seen in AD (Fagarasan et al. Brain Res. 723(1-2):231-4. (1996); Kalaria et al. Neurodegeneration. 5(4):497-503 (1996); Kalaria et al. Neurobiol Aging. 17(5):687-93 (1996); Farlow Am J Health Syst Pharm. 55 Suppl. 2:S5-10 (1998).

Evidence that amyloid β protein (Aβ) deposition causes some forms of AD was provided by genetic and molecular studies of some familial forms of AD (FAD). (See, e.g., Ii Drugs Aging 7(2):97-109 (1995); Hardy PNAS 94(6):2095-7 (1997); Selkoe J. Biol. Chem. 271(31): 18295-8 (1996)). The amyloid plaque buildup in AD patients suggests that abnormal processing of Aβ may be a cause of AD. Aβ is a peptide of 39 to 42 amino acids and is the core of senile plaques is observed in all Alzheimer cases. If abnormal processing is the primary cause of AD, then familial Alzheimer's disease (FAD) mutations that are linked (genetically) to FAD may induce changes that, in one way or another, foster Aβ deposition. There are 3 FAD genes known so far (Hardy et al. Science 282:1075-9 (1998); Ray et al. (1998)). Mutations in these FAD genes can result in increased Aβ deposition.

The first of the 3 FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ₄₂, both in model transfected cells and transgenic animals. The other two FAD genes code for presenilin 1 and 2 (PS1, PS2) (Hardy PNAS 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP genes, and all of them also cause FAD with 100% penetrance. Similar to APP mutants, studies have demonstrated that PS1 and PS2 mutations shift APP metabolism, resulting in elevated Aβ₄₂ production (in vitro and in vivo.)

Cycloxygenases (COX, COX-1, and/or COX-2) are major Alzheimer's disease drug targets due to the intimate association of inhibition of prostaglandin synthesis with NSAIDs use, whose primary target are cycloxygenases, and a reduced risk of developing Alzheimer's disease. COX-2 specific inhibitors are attractive candidates for long-term drug use since they do not inhibit COX-1. COX-2 inhibitors appear to be less-toxic than the non-selective NSAIDS which inhibit COX-1 and/or COX-2. At the 8^{th} international conference on Alzheimer's disease and related disorders, it was reported that rofecoxib, a COX-2 selective NSAID, at 25 mg daily, failed to show efficacy for treating AD. Naproxen, another NSAID, in the same trial failed to show efficacy in Alzheimer's treatment. See Aisen et al. JAMA J289:2819-26 (2003). These authors concluded that the results with naproxen and rofecoxib do not support the use of NSAIDS for the treatment of mild-to-moderate AD. Conversely, it was reported recently that rofecoxib, provides neuroprotection in an in vivo Alzheimer's disease excitotoxic model system (Scali et al. Neuroscience 117:909-919 (2003).

Aβ formation is another target for affecting Alzheimer's disease progression since Aβ amyloid plaques are a central pathological hallmark of the disease. Recently, it was suggested that certain NSAIDs are capable of lowering the level of Aβ₄₂. U.S. patent application 2002/0128319 to Koo et al. discloses the use of an Aβ₄₂ lowering amount of NSAID for treating Alzheimer's disease. R-Flurbiprofen, which negligibly inhibits COX activity, was shown in Koo et al. to lower Aβ₄₂ in a transgenic mouse model and CHO cells. The hope is that by lowering the level of Aβ₄₂, the formation of the amyloid plaques central to the disease would be retarded.

A recent clinical trial using a therapy designed to eliminate Aβ plaques from disease patients failed despite strong evidence of efficacy in animal models (Pieffer et al. Science 298:1379 (2002)). The Aβ-lowering therapy that worked in animal models caused serious problems in humans. In view of the clinical studies, Atwood et al. (Science 299:1014 (2003)) noted that “Mounting evidence indicates that this deposition of amyloid-β may be a neuroprotective response to injury” and “These results demonstrate yet again the futility of removing a protein, amyloid-β, which has ubiquitous tissue expression, without first understanding its function(s).” Additionally, secretase inhibitors, which were designed to alter processing of APP, have turned out to be toxic compounds not likely to be suitable for chronic human use. Thus, it is not clear if reducing Aβ or Aβ₄₂ is a realistic treatment/prevention option. Indeed, as noted recently, mutations in PS-1 associated with AD may cause the disease not through altering Aβ processing but rather by affecting calcium homeostasis (Mattson, Nature 442:385-386 (2003)).

Several epidemiological studies have reported an association between long-term use of NSAIDs, such as ibuprofen and aspirin, with reduced risk for certain malignancies and neurodegenerative processes characterized by dementia of the Alzheimer's type. A variety of explanations have been given for the reduced cancer and Alzheimer's disease (AD) risk associated with long-term NSAID use. The primary action of NSAIDs appears to be inhibition of cyclooxygenase (COX) activity. Thus, a leading hypothesis is that NSAIDs reduce risk for certain cancers and Alzheimer's disease by affecting the COX enzymes. Other explanations include mediation of apoptosis, modulation of growth factors, and modulation of the nuclear factor kappa B pathway (NF-κB).

U.S. Pat. No. 5,192,753 to Rogers et al. discloses the use of NSAIDs to treat Alzheimer's disease through the inhibition of cyclooxygenase and therefore inhibition of prostaglandin synthesis. U.S. Pat. No. 5,643,960 to Brietner et al. discloses the use of COX inhibiting NSAIDs to delay the onset of Alzheimer's symptoms. U.S. Pat. No. 6,025,395 to Brietner et al. relates to the use of COX inhibiting NSAIDs.

Flurbiprofen is a racemic non-steroidal anti-inflammatory drug (NSAID) having a chemical name of (R,S)-(2-fluoro-biphenylyl) propionic acid. 50 milligram (mg) and 100 mg flurbiprofen tablets are marketed as ANSAID® and FROBEN® for the treatment of chronic inflammatory disease.

The literature has described a variety of R-flurbiprofen containing compositions. Brune et al., J. Clin. Pharmacol., 32:944-952 (1992) discloses the use of tablets containing 50 mg of R-flurbiprofen. Jerussi et al., J. Clin. Pharmacol., 32:944-952 (1992) describe the use of 100 mg b.i.d. R-flurbiprofen in investigating gastroduodenal tolerance. Lotsch et al., Bri. J Clin. Pharm., 40:339-346 (1995) describe the use 50 mg and 100 mg doses of R-flurbiprofen in pain related chemo-somatosensory evoked potentials in human subjects. The authors concluded that R-flurbiprofen, at these doses, produced an analgesic effect. Geisslinger et al., Br. J. Clin. Pharmacol., 37(4):392-4 (1994) discloses the use of 50 mg R-flurbiprofen for examining the disposition of single enantiomers in humans. Oelkers et al., Br. J. Clin. Pharmacol. 43(2): 145-53 (1997) discloses the use of 75 mg R-flurbiprofen for studying its effects and disposition in blister fluid and human serum. U.S. Pat. No. 5,206,029 to Brune et al. discloses medicaments, containing 10 to 100 mg doses of previously separated flurbiprofen enantiomers in a ratio of 99.5:.5% to 0.5:99.5%, which are effective for treating pain and inflammatory conditions. U.S. Pat. No. 5,200,198 to Geisslinger et. al. discloses a medicament, containing 10 to 100 mg doses of substantially pure R-flurbiprofen and mixtures containing up to 40% S-enantiomer, that are effective for treating pain and inflammatory conditions.

Statins have also been implicated as potential Alzheimer disease therapeutics by retrospective epidemiological studies. See Petanceska et al., J. Mol. Neurosci., 19:155-61 (2002). These retrospective studies indicate that statin users have a lower prevalence of developing Alzheimer's disease. Since many different explanation can account for the lower prevalence of Alzheimer's disease in statin users aside from the use of statin and combined with the fact that no statins have been approved for an Alzheimer's disease indication, it is not certain if (and how/when) they can be used to treat Alzheimer's disease.

In the United States alone, four million adults suffer from Alzheimer's disease (AD). Not only is Alzheimer's disease significantly impacting the lives of countless families today, it is threatening to become even more of a problem as the baby boom generation matures. The economic burden of AD is estimated to cost over $100 billion a year and the average lifetime cost per patient is estimated to be $174,000. Unfortunately, there is no cure available for AD. Of the five drugs currently being used in the US for the treatment of AD, four of them—tacrine (Cognex®), donepezil (Aricept®), rivastigmine (Exelon®), and galantamine (Reminyl®)—are inhibitors of acetylcholinesterase. Another drug, memantine, was recently approved for treating moderate-to-severe AD. More recently it was reported that memantine showed efficacy in treating mild-to-moderate AD. Memantine is a NMDA receptor antagonist.

The drugs currently used for treating AD, including memantine and the acetylcholine esterase inhibitors, are marginally efficacious and have undesirable side-effects. Thus, there is a large unmet need for better and safer drugs.

### SUMMARY OF THE INVENTION

The invention generally relates to compositions and therapeutic treatments for neurodegenerative disorders. More specifically, the invention provides a composition for treating and delaying the onset of neurodegenerative disorders. The composition of the invention has one or more HMG-CoA-reductase inhibitors and one or more R—NSAIDs and optionally one or more pharmaceutically acceptable carriers. The method of the invention involves administering to an individual in need of treatment with an effective amount of one or more HMG-CoA reductase inhibitors and one ore more R—NSAIDs.

In a first embodiment, the invention provides a composition comprising one or more HMG-CoA reductase inhibitors (including pharmaceutically acceptable salts and esters thereof) and one or more R—NSAIDs (including pharmaceutically acceptable salt and esters thereof). In one aspect of this embodiment, the HMG-CoA reductase inhibitor is a statin. In another aspect of this embodiment, the HMG-CoA reductase inhibitor is a statin selected from the group consisting of atorvastatin, simvastatin, lovastatin, fluvastatin, pravastatin, cerivastatin, rosuvastatin, and pitavastatin. In one aspect of this embodiment the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In yet another aspect of this embodiment, the HMG-CoA reductase inhibitor is selected from the group consisting of atorvastatin, simvastatin, lovastatin, fluvastatin, pravastatin, cerivastatin, rosuvastatin, and pitavastatin and the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac. In still another aspect of this embodiment, the R—NSAID is R-flurbiprofen or a pharmaceutically acceptable salt or ester thereof. In another aspect, the R—NSAID is R-flurbiprofen or a pharmaceutically acceptable salt or ester thereof and the HMG-CoA reductase inhibitor is selected from the group consisting of atorvastatin, simvastatin, lovastatin, fluvastatin, pravastatin, cerivastatin, rosuvastatin, and pitavastatin. The invention further provides compositions having R-flurbiprofen and atorvastatin; R-flurbiprofen and simvastatin; R-flurbiprofen and lovastatin; R-flurbiprofen and fluvastatin; R-flurbiprofen and pravastatin; R-flurbiprofen and cerivastatin; R-flurbiprofen and rosuvastatin; or R-flurbiprofen and pitavastatin. The compositions of this embodiment can provide the two components together in a single dose with a pharmaceutically acceptable carrier.

In a second embodiment, the invention provides a method for treating neurodegenerative disorders. According to the method of this embodiment, an effective amount of one or more R—NSAIDs (including pharmaceutically acceptable salts and esters thereof) and one or more HMG-CoA reductase inhibitors (including pharmaceutically acceptable salts and esters thereof) is administered to an individual in need of such treatment. The individual in need of treatment can have a neurodegenerative disorder, a predisposition to a neurodegenerative disorder, and/or desire prophylaxis against neurodegenerative disorders. In one aspect of this embodiment, the effective amount of the one or more R—NSAIDs and one or more HMG-CoA reductase inhibitors is capable of reducing at least one symptom of the neurodegenerative disorder. In another aspect, for individuals desiring prophylaxis against a neurodegenerative disorder, the effective amount of the one or more R—NSAIDs and one or more HMG-CoA reductase inhibitors, is capable of preventing an increase (or rate of increase) in at least one symptom of the neurodegenerative disorder. In one aspect of this method, the one or more HMG-CoA reductase inhibitors are a statin. In another aspect of this method, the one or more HMG-CoA reductase inhibitors are a statin selected from the group consisting of atorvastatin, simvastatin, lovastatin, fluvastatin, pravastatin, cerivastatin, rosuvastatin, and pitavastatin. In one aspect of this method, the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, or pharmaceutically acceptable salts or esters thereof. In yet another aspect of this method, the HMG-CoA reductase inhibitor is selected from the group consisting of atorvastatin, simvastatin, lovastatin, fluvastatin, pravastatin, cerivastatin, rosuvastatin, and pitavastatin and the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, or a pharmaceutically acceptable salt or ester thereof. In still another aspect of this method, the R—NSAID is R-flurbiprofen. In another aspect of this method the R—NSAID is R-flurbiprofen and the HMG-CoA reductase inhibitor is selected from the group consisting of atorvastatin, simvastatin, lovastatin, fluvastatin, pravastatin, cerivastatin, rosuvastatin, and pitavastatin. The method of the invention further provides for the treatment or prophylaxis of neurodegenerative disorders by administering an effective amount of R-flurbiprofen and atorvastatin; R-flurbiprofen and simvastatin; R-flurbiprofen and lovastatin; R-flurbiprofen and fluvastatin; R-flurbiprofen and pravastatin; R-flurbiprofen and cerivastatin; R-flurbiprofen and rosuvastatin; or R-flurbiprofen and pitavastatin. In a preferred aspect of this method, the neurodegenerative disease is selected from the group consisting of Alzheimer's disease, dementia, mild cognitive impairment, and tauopathies (e.g., corticobasal degeneration, frontotemporal dementia with Parkinsonism linked to chromosome 17, and progressive supranuclear palsy). In another preferred embodiment, the invention provides a method for the treatment or prophylaxis of Alzheimer's disease through the administration of an effective amount of R-flurbiprofen and atorvastatin; R-flurbiprofen and simvastatin; R-flurbiprofen and lovastatin; R-flurbiprofen and fluvastatin; R-flurbiprofen and pravastatin; R-flurbiprofen and cerivastatin; R-flurbiprofen and rosuvastatin; or R-flurbiprofen and pitavastatin.

In a third embodiment, the invention provides a method of reducing amyloid β₄₂ (Aβ₄₂) protein levels in a mammal, for example in brain or in a body fluid such as CSF or plasma or blood. In particular, the method relates to reducing, lowering, or preventing an increase in Aβ₄₂ protein levels, in an individual in need of such treatment, by administering to the individual an effective amount of one or more R—NSAIDs (including pharmaceutically acceptable salts and esters thereof) and one or more HMG-CoA reductase inhibitors (including pharmaceutically acceptable salts and esters thereof). The individual in need of treatment can have a neurodegenerative disorder, a predisposition to a neurodegenerative disorder, and/or a desire for prophylaxis against neurodegenerative disorders, where the disorder is characterized by increased Aβ₄₂ protein levels. In one aspect, the effective amount is an amount of one or more R—NSAIDs and one or more HMG-CoA reductase inhibitors sufficient for reducing Aβ₄₂ protein levels. In a preferred aspect, the effective amount is an amount of one or more R—NSAIDs and one or more HMG-CoA reductase inhibitors sufficient for reducing Aβ₄₂ protein levels and lowering cholesterol levels, particularly LDL cholesterol levels. In another aspect, for individuals desiring prophylaxis against a neurodegenerative disorder, the effective amount is an amount of one or more R—NSAIDs and one or more HMG-CoA reductase inhibitors, sufficient for preventing an increase in Aβ₄₂ protein levels or an increase in the rate of Aβ₄₂ increase. In one aspect of this method, the HMG-CoA reductase inhibitor is a statin. In another aspect of this method, the HMG-CoA reductase inhibitors is a statin selected from the group consisting of atorvastatin, simvastatin, lovastatin, fluvastatin, pravastatin, cerivastatin, rosuvastatin, and pitavastatin. In one aspect of this method, the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac. In yet another aspect of this method, the HMG-CoA reductase inhibitor is selected from the group consisting of atorvastatin, simvastatin, lovastatin, fluvastatin, pravastatin, cerivastatin, rosuvastatin, and pitavastatin and the R—NSAID is selected from the group consisting of R-flurbiprofen, R-ketoprofen, R-ketorolac, R-naproxen, R-tiaprofenic acid, R-suprofen, R-carprofen, R-pirprofen, R-indoprofen, R-benoxaprofen, and R-etodolac. In still another aspect of this method, the R—NSAID is R-flurbiprofen. In another aspect of this method, the R—NSAID is R-flurbiprofen and the HMG-CoA reductase inhibitor is selected from the group consisting of atorvastatin, simvastatin, lovastatin, fluvastatin, pravastatin, cerivastatin, rosuvastatin, and pitavastatin. The method of the invention further provides for the treatment or prophylaxis of neurodegenerative disorders with an Aβ₄₂ protein lowering effective amount of R-flurbiprofen and atorvastatin; R-flurbiprofen and simvastatin; R-flurbiprofen and lovastatin; R-flurbiprofen and fluvastatin; R-flurbiprofen and pravastatin; R-flurbiprofen and cerivastatin; R-flurbiprofen and rosuvastatin; or R-flurbiprofen and pitavastatin. In a preferred aspect of this method, the neurodegenerative disease is selected from the group consisting of Alzheimer's disease, dementia, and mild cognitive impairment. In another preferred embodiment, the invention provides a method for the treatment or prophylaxis of Alzheimer's disease through the administration of an Aβ₄₂ protein lowering effective amount of R-flurbiprofen and atorvastatin; R-flurbiprofen and simvastatin; R-flurbiprofen and lovastatin; R-flurbiprofen and fluvastatin; R-flurbiprofen and pravastatin; R-flurbiprofen and cerivastatin; R-flurbiprofen and rosuvastatin; or R-flurbiprofen and pitavastatin.

In fourth embodiment, the invention provides compositions and a method for treating and/or preventing neurodegenerative disorders by administering, to an individual in need of such treatment, an effective amount of one or more R—NSAIDs (including pharmaceutically acceptable salts and esters thereof), one or more statins such as atorvastatin, simvastatin, lovastatin, fluvastatin, pravastatin, cerivastatin, rosuvastatin, and pitavastatin (including pharmaceutically acceptable salts and esters thereof) , and one or more compounds selected from the group consisting of secretase inhibitors, acetylcholine esterase inhibitors, GABA-A alpha 5 inverse agonists, and antioxidants (or pharmaceutically acceptable salts and esters thereof). The combination can be administered simultaneously or separately.

In a fifth embodiment, the invention provides a method of lowering Aβ₄₂ levels to a greater extent than inhibiting COX-1, COX-2, or a combination thereof. In particular, the method of this embodiment involves administering to a patient, in need of treatment, an effective amount of one or more R—NSAIDs (including pharmaceutically acceptable salts and esters thereof) and one or more HMG-CoA reductase inhibitors (including pharmaceutically acceptable salts and esters thereof). According to one aspect of this embodiment, the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac. According to another aspect of this embodiment, the HMG-CoA reductase inhibitor is a statin selected from the group consisting of atorvastatin, simvastatin, lovastatin, fluvastatin, pravastatin, cerivastatin, rosuvastatin, and pitavastatin. In another aspect of this embodiment, the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and the HMG-CoA reductase inhibitor is a statin selected from the group consisting of atorvastatin, simvastatin, lovastatin, fluvastatin, pravastatin, cerivastatin, rosuvastatin, and pitavastatin. In a preferred aspect of this embodiment, the R—NSAID is R-flurbiprofen and the HMG-CoA reductase inhibitor is selected from the group consisting of atorvastatin, simvastatin, lovastatin, fluvastatin, pravastatin, cerivastatin, rosuvastatin, and pitavastatin. The method of this embodiment involves the lowering of Aβ₄₂ levels while not substantially affecting the activity of COX-1, COX-2, or both COX-1 and COX-2. Thus, the amount that is administered is effective for lowering Aβ₄₂ levels and does not substantially inhibit COX-1, COX-2, or both COX-1 and COX-2. For example, the effective amount can be above the ED₅₀ (the dose therapeutically effective in 50% of the population) for Aβ₄₂ lowering, and below the ED₅₀ for COX inhibition. Another example is a sufficiently small amount of compound so that inhibition of at least one COX activity is negligible and Aβ₄₂ levels are reduced. The method of this embodiment can be used to treat and/or prevent Alzheimer's disease. The method of this embodiment can also be used to treat and/or prevent MCI, dementia, and other neurodegenerative disorders.

In a sixth embodiment, the invention provides a compound having one or more statins conjugated to one or more NSAIDs. The statin can be directly covalently conjugated to the NSAID or may be attached through a linker group. This embodiment provides compounds having a general structure of X—SP-L-SP-L-SP—Y where X is one or more statin(s) or HMG-CoA reductase inhibitor(s), each SP is a spacer that is independent of one another and may or may not be present, each L is a linker that is independent of one another and may or may not be present, and Y is one or more NSAID(s), preferably an R—NSAID. In one aspect of this embodiment, the statin is selected from the group consisting of atorvastatin, simvastatin, lovastatin, fluvastatin, pravastatin, cerivastatin, rosuvastatin, and pitavastatin. In another aspect of this embodiment, the NSAID is flurbiprofen or R-flurbiprofen. In yet another aspect of this embodiment, the NSAID is an Aβ₄₂ lowering agent.

In a seventh embodiment, the invention provides compositions and a therapeutic method comprising administering, to an individual in need of such treatment, an effective amount of one or more R—NSAIDs (including pharmaceutically acceptable salts and esters thereof) capable of lowering Aβ₄₂ levels, and an inhibitor of intestinal cholesterol absorption (e.g., an inhibitor of aminopeptidase N (also known as (alanyl)aminopeptidase, or leukemia antigen CD13) that blocks endocytosis of cholesterol-rich membrane microdomains, thereby limiting intestinal cholesterol absorption). Preferably, the composition comprises an effective amount of R-flurbiprofen or a pharmaceutically acceptable salt or ester thereof, and ezetimibe (the active ingredient of Zetia® by Schering-Plough) or pharmaceutically acceptable salt thereof. In another aspect of this embodiment, the composition further include one or more statins such as atorvastatin, simvastatin, lovastatin, fluvastatin, pravastatin, cerivastatin, rosuvastatin, and pitavastatin (including pharmaceutically acceptable salts and esters thereof).

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

### 1.0. INTRODUCTION

The invention provides compositions and therapeutic treatments for neurodegenerative disorders, e.g., Alzheimer's disease, MCI, Down's syndrome, and tauopathies (e.g., corticobasal degeneration, frontotemporal dementia with Parkinsonism linked chromosome 17, and progressive supranuclear palsy, etc.). Specifically, the invention provides a composition, for treating and delaying the onset of neurodegenerative disorders, having one or more HMG-CoA-reductase inhibitors (including pharmaceutically acceptable salts and esters thereof) and one or more R—NSAIDs (including pharmaceutically acceptable salts and esters thereof). The invention provides a method that involves treating an individual in need of treatment with an effective amount of one or more HMG-CoA reductase inhibitors and one or more R—NSAIDs. The method of the invention can involve co-administering the one or more HMG-CoA reductase inhibitors and the one or more R—NSAIDs, or the one or more HMG-CoA reductase and the one or more R—NSAIDs can be administered to the same individual at different times and/or by different routes of administration. For example, the HMG-CoA reductase inhibitor can be administered in the morning and the R—NSAID can be administered in the evening. Advantageously, the combination of R—NSAID and statin can be administered together as a conjugate described herein. Without wishing to be bound by theory, it is believed that the NSAID-statin conjugates have unexpected properties particularly useful for the treatment and prophylaxis of neurodegenerative disease like dementia, mild cognitive impairment, and/or Alzheimer's disease.

The invention provides a composition having one or more HMG-CoA reductase inhibitors (including pharmaceutically acceptable salts and esters thereof) and one or more R—NSAIDs (including pharmaceutically acceptable salts and esters thereof). The HMG-CoA reductase inhibitors used in the invention can be a statin. Preferred HMG-CoA reductase inhibitors are statins selected from the group consisting of atorvastatin, simvastatin, lovastatin, fluvastatin, pravastatin, cerivastatin, rosuvastatin, and pitavastatin, and pharmaceutically acceptable salts and esters thereof. Preferred R—NSAIDs are selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac (including pharmaceutically acceptable salts and esters thereof). It is contemplated that nitrosylated and nitrosated NSAIDs (and R—NSAIDs) can also be used in the methods of the invention (see, e.g., U.S. Pat. Nos. 6,593,347; 5,703,073; and PCT application WO 94/12463 which are herein incorporated by reference in their entirety). Preferably, the HMG-CoA reductase inhibitor is selected from the group consisting of atorvastatin, simvastatin, lovastatin, fluvastatin, pravastatin, cerivastatin, rosuvastatin, and pitavastatin and pharmaceutically acceptable salts and esters thereof, and the R—NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts and esters thereof. A preferred composition of the invention has R-flurbiprofen and one or more HMG-CoA reductase inhibitor. Another preferred composition has R-flurbiprofen or a pharmaceutically acceptable salt or ester thereof and one or more HMG-CoA reductase inhibitor selected from the group consisting of atorvastatin, simvastatin, lovastatin, fluvastatin, pravastatin, cerivastatin, rosuvastatin, and pitavastatin, and pharmaceutically acceptable salts and esters thereof. The invention further provides composition having R-flurbiprofen or a pharmaceutically acceptable salt thereof and atorvastatin or a pharmaceutically acceptable salt thereof; R-flurbiprofen or a pharmaceutically acceptable salt thereof, and simvastatin or a pharmaceutically acceptable salt thereof; R-flurbiprofen or a pharmaceutically acceptable salt thereof, and lovastatin; R-flurbiprofen and fluvastatin; R-flurbiprofen or a pharmaceutically acceptable salt thereof, and pravastatin or a pharmaceutically acceptable salt thereof; R-flurbiprofen or a pharmaceutically acceptable salt thereof, and cerivastatin or a pharmaceutically acceptable salt thereof; R-flurbiprofen or a pharmaceutically acceptable salt thereof, and rosuvastatin or a pharmaceutically acceptable salt thereof; or R-flurbiprofen or a pharmaceutically acceptable salt thereof, and pitavastatin or a pharmaceutically acceptable salt thereof. Without wishing to be bound by any theory, it is believed the compositions of the invention are unexpectedly useful for treating neurodegenerative disorders and exhibit synergistic and/or unexpected effects when used in combination for treating neurodegenerative disorders.

The present invention also provides a pharmaceutical composition comprising a therapeutically effective amount of one or more R—NSAIDs (including pharmaceutically acceptable salts and esters thereof) capable of lowering Aβ₄₂ levels (see U.S. application Publication 20020128319), and a therapeutically effective amount of an inhibitor of intestinal cholesterol absorption (e.g., an inhibitor of aminopeptidase N (also known as (alanyl)aminopeptidase, or leukemia antigen CD13) that blocks endocytosis of cholesterol-rich membrane microdomains, thereby limiting intestinal cholesterol absorption). Preferably, the composition comprises a therapeutically effective amount of R-flurbiprofen or a pharmaceutically acceptable salt or ester thereof, and ezetimibe (the active ingredient of Zetia® by Schering-Plough) or pharmaceutically acceptable salt thereof. The composition optionally further comprises a therapeutically effective amount (effective to lower blood cholesterol level) one or more statins such as atorvastatin, simvastatin, lovastatin, fluvastatin, pravastatin, cerivastatin, rosuvastatin, and pitavastatin (including pharmaceutically acceptable salts and esters thereof).

The pathological hallmarks of Alzheimer's disease are most prevalent in the brain regions involved in higher cognitive functions. These features include a marked loss of neurons and synapses, numerous extracellular neuritic (senile) plaques and intracellular neurofibrillary tangles. The plaques are formed by a core of amyloid material surrounded by a halo of dystrophic neurites. The major component of the core is a peptide of 37 to 43 amino acids in length called the amyloid beta protein (Aβ), the major forms being Aβ₄₀ and Aβ₄₂. The tangles are formed by paired helical filaments, the major component of which is a hyperphosphorylated form of the microtubule-associated protein tau (τ). A large body of evidence suggests that the metabolism of APP and the generation of the Aβ peptide are central in AD pathogenesis. In fact, APP metabolism is regarded as the biochemical link between the pathology and genetics of AD. In addition, lowering Aβ₄₂ leads to clearance of tau pathology and treatment of tauopathies.

Accordingly, in a preferred embodiment, the invention provides methods for lowering or preventing an increase in Aβ₄₂ levels in an individual in need of such treatment. It is believed that by lowering the amounts of Aβ₄₂ in an individual by administering an Aβ₄₂ lowering effective amount of one or more R—NSAIDs (or pharmaceutically acceptable salts and esters thereof) and one or more HMG-CoA reductase inhibitors (or pharmaceutically acceptable salts and esters thereof), as described herein, Alzheimer's disease, dementia, and mild cognitive impairment can be treated, or the onset of such diseases and disorders can be delayed. Generally, the method relates to the idea that administering, to an individual, an effective amount of one or more R—NSAIDs and one or more HMG-CoA reductase inhibitors can lower Aβ₄₂ levels. Thus, diseases characterized by increased levels of Aβ₄₂, can be treated or prevented with the methods of this embodiment which are designed to lower Aβ₄₂ or prevent an increase in Aβ₄₂.

While not wishing to be bound by any theory, it is contemplated that administration of one or more R—NSAIDs, e.g., R-flurbiprofen, and one or more HMG-CoA reductase inhibitors, e.g., atorvastatin, simvastatin, lovastatin, fluvastatin, pravastatin, cerivastatin, rosuvastatin, and pitavastatin, can act in vivo, synergistically to treat and/or prevent Alzheimer's disease, dementia, MCI by lowering the amount of Aβ₄₂ that is present or would be present in the absence of such treatment. Amyloid β polypeptides are derived from amyloid precursor proteins (APPs). A variety of amyloid β polypeptides are known including Aβ₃₄, Aβ₃₇, Aβ₃₈, Aβ₃₉, and Aβ₄₀. Increased Aβ₄₂ levels are associated with Alzheimer's disease, dementia, MCI. Thus, by lowering the amounts of Aβ₄₂, a treatment is provided for combating Alzheimer's disease and/or MCI.

According to a preferred embodiment, the invention provides a method of lowering Aβ₄₂ levels to a greater extent than inhibiting COX-1, COX-2, or a combination thereof. In particular, the method of this embodiment comprises administering, to a patient in need of treatment, an effective amount of an R—NSAID (or pharmaceutically acceptable salts and esters thereof), e.g., R-flurbiprofen, and one or more HMG-CoA reductase inhibitors (or pharmaceutically acceptable salts and esters thereof) such as atorvastatin, simvastatin, lovastatin, fluvastatin, pravastatin, cerivastatin, rosuvastatin, and pitavastatin, and pharmaceutically acceptable salts and esters thereof, wherein the effective amount of composition is capable of lowering Aβ₄₂, while not substantially affecting or inhibiting the activity of at least one isoform of COX. Thus, the method of this embodiment involves the lowering of Aβ₄₂ levels while not substantially inhibiting the activity of COX-1, COX-2, or both COX-1 and COX-2. The method of this embodiment can be used to treat and/or prevent Alzheimer's disease, MCI, dementia, and/or other neurodegenerative disorders. In one aspect of this embodiment, the effective amount of the one or more R—NSAIDs, e.g., R-flurbiprofen and one or more HMG-CoA reductase inhibitors, such as atorvastatin, simvastatin, lovastatin, fluvastatin, pravastatin, cerivastatin, rosuvastatin, and pitavastatin, reduces Aβ₄₂ levels or production of Aβ₄₂ by at least 1, 2, 5, 10, 15, 20, 25, 30, 40, or 50 or more percent while inhibiting COX-1, COX-2, or both COX-1 and COX-2 by less than 1, 2, 5, 10, 15, 20, 25, 30, 40, 50, 60, 70, 80, or 90 percent. In a preferred aspect of this embodiment, the effective amount of the R—NSAID, e.g., R-flurbiprofen, and one or more HMG-CoA reductase inhibitors, such as atorvastatin, simvastatin, lovastatin, fluvastatin, pravastatin, cerivastatin, rosuvastatin, and pitavastatin, lowers Aβ₄₂ by at least 5 percent while not substantially inhibiting COX-1, COX-2, or both COX-1 and COX-2 activity or levels. In another preferred aspect of this embodiment, the effective amount of the R—NSAID, e.g., R-flurbiprofen, and one or more HMG-CoA reductase inhibitors such as atorvastatin, simvastatin, lovastatin, fluvastatin, pravastatin, cerivastatin, rosuvastatin, and pitavastatin, that is administered to an individual is such that it lowers Aβ₄₂ levels, and does not inhibit COX activity to a significant extent, e.g., the amount administered is below the in vivo IC₅₀ value for COX-1, COX-2 or both COX-1 and COX-2 and above the in vivo IC₅₀ value for Aβ₄₂ lowering activity. As used in this context, IC₅₀ refers to the concentration of compound or composition sufficient to inhibit COX activity by 50% (COX-1, COX-2, or both COX-1 and COX-2) or reduce Aβ₄₂ levels (or rates of production) by 50%. An “effective amount” according to this preferred aspect of this embodiment, can also be viewed in terms of ED₅₀ parameters, binding constants, dissociation constants, and other pharmacological parameters, e.g., the amount administered is below the ED₅₀ value for COX-1, COX-2 or both COX-1 and COX-2 and above the ED₅₀ value for Aβ₄₂. It is noted that the effective amount of the compound does not necessarily have to be above an IC₅₀ or ED₅₀ for Aβ₄₂ lowering and below the IC₅₀ or ED₅₀ for COX inhibition. That is, the “effective amount” can be at some intermediate value such that Aβ₄₂ levels (or rates of production) are lowered to a greater extent than inhibition of COX-1, COX-2 or both COX-1 and COX-2. In one aspect, the method of this embodiment is thought to avoid the liability of adverse side effects associated with COX-1 and COX-2 inhibitors.

In another embodiment, the invention provides a method of lowering the Aβ₄₂ level and increasing the Aβ₃₈ level, while not affecting the Aβ₄₀ level in a human in a body fluid, e.g., CSF or blood or plasma. The method of this embodiment comprises administering, to an individual in need of such treatment, an effective amount of an R—NSAID (or pharmaceutically acceptable salts and esters thereof), e.g., R-flurbiprofen, and one or more HMG-CoA reductase inhibitors such as atorvastatin, simvastatin, lovastatin, fluvastatin, pravastatin, cerivastatin, rosuvastatin, and pitavastatin, and pharmaceutically acceptable salts and esters thereof. The method according to this embodiment is useful for treating and delaying the onset of Alzheimer's disease. It is also contemplated that the method of this embodiment is useful for treating and delaying the onset of other disorders such as MCI, dementia, and other neurodegenerative disorders. The Aβ₄₂ lowering method of this embodiment can also increase the levels of other Aβ proteins smaller than Aβ₄₀, including Aβ₃₄, Aβ₃₇, Aβ₃₈, and Aβ₃₉.

In another embodiment, the invention relates to a method of delaying the onset Alzheimer's disease. According to this embodiment, a method for delaying the onset of Alzheimer's disease is provided which comprises administering, to an individual in need of such treatment, an effective amount of an R—NSAID (or pharmaceutically acceptable salts and esters thereof) , e.g., R-flurbiprofen (or pharmaceutically acceptable salts and esters thereof), and one or more HMG-CoA reductase inhibitors such as atorvastatin, simvastatin, lovastatin, fluvastatin, pravastatin, cerivastatin, rosuvastatin, and pitavastatin (or pharmaceutically acceptable salts and esters thereof). The method of this embodiment is useful for delaying the onset of Alzheimer's disease, delaying the onset of one or more symptoms of Alzheimer's disease, and/or the progression of the disease.

The invention provides, in yet another embodiment, a method of decreasing cognitive decline in a patient in need of such treatment. The method of this embodiment involves treating an individual desiring (or needing) a slowing or decrease in decline in cognitive function, with an effective amount of an R—NSAID (i.e., R-flurbiprofen or a pharmaceutically acceptable salt or ester thereof) and a statin (or pharmaceutically acceptable salts and esters thereof). Preferably the statin is atorvastatin, simvastatin, lovastatin, fluvastatin, pravastatin, cerivastatin, rosuvastatin, or pitavastatin. Alternatively, the individual needing a slowing or decrease in decline in cognitive function is administered a conjugate of the invention having one or more R—NSAIDs (i.e., R-flurbiprofen) conjugated to one or more statins.

In one example, a patient suspected of having mild-to-moderate Alzheimer's disease is identified using diagnostic techniques readily available to the skilled practitioner. The patient is then administered, on a daily basis, or twice daily basis, an Alzheimer's disease therapeutically effective amount of R-flurbiprofen and a statin. Preferably the statin is atorvastatin, simvastatin, lovastatin, fluvastatin, pravastatin, cerivastatin, rosuvastatin, or pitavastatin.

In another aspect of the present invention, a method is provided for ameliorating or delaying the onset of a disease or disorder in a patient comprising treating the patient with a therapeutically effective amount of R-flurbiprofen, and an HMG-CoA reductase inhibitor or ezetimibe. An HMG-CoA reductase inhibitor can be a statin such as atorvastatin, simvastatin, lovastatin, fluvastatin, pravastatin, cerivastatin, rosuvastatin, and pitavastatin. As used herein, the phrase “treating . . . with . . . a compound (e.g. R-flurbiprofen)” or paraphrases thereof means either administering the compound to a patient, or administering to a patient the compound or another agent to cause the presence or formation of the compound inside the patient.

It is noted that the combination treatment can be applied to a patient for purposes of treating any suitable diseases and disorders, including but not limited to, dementia, Alzheimer's disease, mild cognitive impairment (MCI), tauopathies (e.g., corticobasal degeneration, frontotemporal dementia with Parkinsonism linked to chromosome 17, and progressive supranuclear palsy), Down's Syndrome, coronary heart disease, hypercholesterolemia, diabetic dyslipidemia, rheumatoid arthritis, inflammatory disorders, chronic kidney diseases, proteinuria and others. Thus, the patient treated can have one of the diseases and disorders requiring treatment, or have two or more of the diseases and disorders. For example, the compositions and combination treatment of the present invention can be particularly desirable if a patient desires treatment (therapeutic treatment and/or delaying the onset) of a first disease selected from dementia, Alzheimer's disease, mild cognitive impairment (MCI), tauopathies (e.g., corticobasal degeneration, frontotemporal dementia with Parkinsonism linked to chromosome 17, and progressive supranuclear palsy), and Down's Syndrome, and a second disease selected from coronary heart disease, hypercholesterolemia, diabetic dyslipidemia, rheumatoid arthritis, inflammatory disorders, chronic kidney diseases. In particular, the compositions and combination treatment of the present invention can be advantageous in the treatment of a patient having, and/or desiring a delay in the onset of, both Alzheimer's disease or MCI, and hypercholesterolemia.

Preferably, the daily dosage of R-flurbiprofen is from about 5 mg to about 2000 mg, more preferably from about 50 mg to about 1800 mg and even more preferably from about 200 to about 1600 mg. Preferably, the daily dosage of statin is as follows: from about 1 mg to 100 mg atorvastatin; from about 0.5 mg to about 100 mg of simvastatin; from about 1 mg to 100 mg of lovastatin; from about 1 mg to about 100 mg of fluvastatin; from about 0.5 mg to about 50 mg pravastatin; from about 0.01 mg to about 1.5 mg cerivastatin; from about 1 mg to about 50 mg rosuvastatin; and from about 1 mg to about 100 mg of pitavastatin. More preferably, the daily dosage of statin is as follows: from about 1 mg to 50 mg atorvastatin; from about 0.5 mg to about 50 mg of simvastatin; from about 1 mg to 50 mg of lovastatin; from about 1 mg to about 50 mg of fluvastatin; from about 0.5 mg to about 30 mg pravastatin; from about 0.01 mg to about 0.5 mg cerivastatin; from about 1 mg to about 30 mg rosuvastatin; and from about 1 mg to about 50 mg of pitavastatin. Even more preferably, the daily dosage of statin is as follows: from about 1 mg to 25 mg atorvastatin; from about 0.5 mg to about 25 mg of simvastatin; from about 1 mg to 25 mg of lovastatin; from about 1 mg to about 25 mg of fluvastatin; from about 0.5 mg to about 15 mg pravastatin; from about 0.01 mg to about 0.25 mg cerivastatin; from about 1 mg to about 20 mg rosuvastatin; and from about 1 mg to about 25 mg of pitavastatin. Preferably, the amount of statin administered is a cholesterol lowering effective amount. A “cholesterol lowering effective amount” refers to an amount that reduces cholesterol in vivo in a human. Many statins have been approved by the US FDA for use to lower cholesterol level. Any of the dosages approved by the US FDA or equivalent dosages are cholesterol lowering effective amount. For ezetimibe, the daily dosage is from 1 to 50 mg, preferably from 2 to 20 mg, e.g., 10 mg. Preferably, cholesterol, particularly LDL cholesterol is lowered by at least 5%, more preferably at least 10%, even more preferably 15%, as compared to the amount of cholesterol present in the subject in the absence of treatment. Alternatively, a conjugate of the invention comprising R-flurbiprofen and a statin selected from atorvastatin, simvastatin, lovastatin, fluvastatin, pravastatin, cerivastatin, rosuvastatin, or pitavastatin is administered to the patient. Similar (i.e., bioequivalent) dosages of total amounts of statin and R-flurbiprofen in the conjugate, as described above, can be used according to this aspect of the invention. Individuals having mild-to-moderate Alzheimer's disease, according to this embodiment, are treated with the above recommended daily doses for 24 weeks or more, preferably 36 weeks or more, even more preferably for one year or more, with the combination of R-flurbiprofen and statin. Desirably, the combination can be formulated in a single dosage form such as a tablet, capsule, or liquid for oral administration. Alternatively, the individual components of the combination (R-flurbiprofen and statin) can be administered separately, i.e., a tablet of R-flurbiprofen and a tablet having the statin. Again, the combination can be administered together as a conjugate.

In a preferred embodiment of the invention, a method for treating Alzheimer's disease is provided which involves administering to a patient an Aβ₄₂ lowering effective amount of a compound (e.g., R-flurbiprofen or a pharmaceutically acceptable salt or ester thereof) and a cholesterol lowering effective amount of statin or ezetimibe (or a LDL cholesterol lowering effective amount of statin or ezetimibe).

In preferred embodiments, a dosage having R-flurbiprofen in an amount of about 400 mg to about 800 mg per dose is included in the combination of the present invention. The dose can be provided twice daily, in a single or multiple dosage units (i.e., tablets or capsules) having about 350 mg R-flurbiprofen, 400 mg R-flurbiprofen, 450 mg R-flurbiprofen, 500 mg R-flurbiprofen, 550 mg R-flurbiprofen, 600 mg R-flurbiprofen, 650 mg R-flurbiprofen, 700 mg R-flurbiprofen, 750 mg R-flurbiprofen, 800 mg R-flurbiprofen, or 850 mg R-flurbiprofen or a pharmaceutically acceptable salt or ester thereof. For example, the dosage unit is 400 mg; thus, a preferred composition of the invention comprises 400 mg R-flurbiprofen and an LDL cholesterol lowering effective amount of statin or ezetimibe, and a carrier or vehicle suitable for oral administration, e.g., in tablets or capsules. Another preferred dose is 800 mg of R-flurbiprofen, and a preferred composition of the invention comprises 400 mg R-flurbiprofen and an LDL cholesterol lowering effective amount of statin or ezetimibe, and a carrier or vehicle suitable for oral administration, e.g., in tablets or capsules. Preferably, the compositions are substantially free of S-flurbiprofen.

Oral administration of a dose, twice daily for at least 4 months, preferably 8 months, and more preferably 1 year, can provide an improvement or lessening of decline in cognitive function, biochemical disease marker progression, and/or plaque pathology.

Desirably, the R-flurbiprofen-containing compositions of the invention are substantially free of the S-stereoisomer of flurbiprofen. In one aspect, substantially free of the S-stereoisomer means at least 90% by weight R-flurbiprofen to 10% by weight or less of S-flurbiprofen of the total flurbiprofen (S+R flurbiprofen) in said pharmaceutical composition. In another aspect, substantially free of the S-stereoisomer means at least 95% by weight R-flurbiprofen to 5% by weight or less of S-flurbiprofen of the total flurbiprofen (S+R flurbiprofen) in the pharmaceutical composition. In yet another aspect, substantially free of the S-stereoisomer means at least 99% by weight R-flurbiprofen to 1% by weight or less of S-flurbiprofen of the total flurbiprofen (S+R flurbiprofen) in the pharmaceutical composition. In yet another aspect, substantially free of the S-stereoisomer means at least 99.9% by weight R-flurbiprofen to 0.1% by weight or less of S-flurbiprofen of the total flurbiprofen (S+R flurbiprofen) in the pharmaceutical composition. In one aspect, a preferred dosage form is a tablet. In another aspect, a preferred dosage form is a capsule. In other aspects, the composition provides an improvement or lessening in decline in biochemical disease marker progression, plaque pathology, quality of life indicators or combinations of any disease parameters.

In preferred embodiments, in the R-flurbiprofen-containing compositions and combination treatment methods of the present invention, R-flurbiprofen or a pharmaceutically acceptable salt or ester thereof is administered in an amount sufficient to result in a plasma Cₘₐₓ of about 20 to about 150 μg per mL, and wherein said individual is known to have, or is suspected of having, AD or MCI. In a more specific embodiment, said plasma Cₘₐₓ is from about 30 to about 95 μg per mL. In another more specific embodiment, said Cₘₐₓ is from about 40 to about 80 μg per mL. In another embodiment, said Cₘₐₓ is between about 100 and about 600 μM. In a more specific embodiment, said plasma Cₘₐₓ is from about 150 to about 380 μM. In another more specific embodiment, said Cₘₐₓ is from about 170 to about 240 μM. In a specific, preferred embodiment, said individual has mild to moderate AD or MCI.

In another embodiment, R-flurbiprofen or a pharmaceutically acceptable salt or ester thereof is administered in an amount sufficient to result in a cerebrospinal fluid R-flurbiprofen Cₘₐₓ of about 0.05 to about 7.5 μg per mL, and wherein said individual is known to have, or is suspected of having, AD or MCI. In another embodiment, said Cₘₐₓ is from about 0.08 to about 4.5 μg per mL. In another embodiment, the R-flurbiprofen or a pharmaceutically acceptable salt or ester thereof is administered in an amount sufficient to result in a cerebrospinal fluid R-flurbiprofen Cₘₐₓ of about 2 to 30 μM; from about 3.2 μM to about 20 μM; or from about 4 μM to about 12 μM.

The time to achieve plasma R-flurbiprofen Cₘₐₓ will depend upon the individual to be treated, but is preferably between 0.70 to 3.75 hours. In various embodiments, the tₘₐₓ (time to Cₘₐₓ) is from about 0.75 to 2.00 hours, or is from about 0.75 hour to about 1.75 hours. For example, tₘₐₓ can be about 2 hours after administration. Preferably, the t_{1/2} (half-life) is from about 3.75 to about 8.5 hours.

Somewhat more time is expected to achieve a cerebrospinal fluid Cₘₐₓ; however, this Cₘₐₓ is expected to be achieved between about 1 hour and about 6 hours after administration of a dose of R-flurbiprofen according to the invention.

R-flurbiprofen levels in the plasma or in the cerebrospinal fluid may be assessed by any art-accepted method. Determination of the concentration of R-flurbiprofen in cerebrospinal fluid may be accomplished as follows. Cerebrospinal fluid containing flurbiprofen and an internal standard, for example, flurbiprofen-D₃, is mixed with mobile phase and centrifuged. The supernatant is then transferred to a 96-well block and an aliquot of extract is injected onto a Micromass Ultima LC-MS-MS equipped with an enantio-selective column. Peak area of the m/z 243→199 flurbiprofen product ion is measured against the peak area of the m/z 246→202 flurbiprofen-D₃ internal standard product ion. Quantification may be performed using a weighted (1/x²) linear least squares regression analysis for each enantiomer generated from fortified plasma standards prepared in bulk and frozen.

The plasma half-life will also depend upon the individual to be treated. Preferably, the plasma half-life of R-flurbiprofen is from about 3.75 to about 8.5 hours. Preferably, administration of a single dose to a fasting subject provides an AUC (area under curve of concentration versus time; total drug exposure) of R-flurbiprofen of from about 200 hr·μg/mL to about 600 hr·μg/mL. Also preferably, the R-flurbiprofen in the compositions and methods of the present invention is such that in repeating administrations an AUC₁₂ (area under curve of concentration in a 12-hour window, i.e., total drug exposure in a 12-hour window) is from about 200 hr·μg/mL to about 450 hr·μg/mL. Thus, in one embodiment, a composition of the present invention is administered to an individual having one or more indications of Alzheimer's disease or MCI, to achieve a plasma concentration in said individual of R-flurbiprofen of between 30 and 95 μg per mL by no more than 3.75 hours after administration. In a specific embodiment, said plasma concentration is achieved within 1.75 hours after administration. In another specific embodiment, said plasma concentration is achieved between 0.75 hours and 3.75 hours after administration. In another specific embodiment, said plasma concentration is between 40 and 80 μg per mL. In another specific embodiment, said individual is an individual that has been diagnoses having mild to moderate Alzheimer's disease or MCI or that would be diagnosed as having mild to moderate Alzheimer's disease or MCI according to a test of cognition.

In one embodiment, the R-flurbiprofen-containing compositions of the present invention are administered for a method of administering R-flurbiprofen to an individual, wherein said R-flurbiprofen is administered in an amount sufficient to result in a plasma Cₘₐₓ of about 35 to about 50 μg per mL, and wherein said individual is known to have, or is suspected of having, AD. In a more specific embodiment, said plasma Cₘₐₓ is from about 38 to about 48 μg per mL. In another more specific embodiment, said Cₘₐₓ is from about 39 to about 46 μg per mL. In another embodiment, the invention provides for a method of administering R-flurbiprofen to an individual, wherein said R-flurbiprofen is administered in an amount sufficient to result in a plasma Cₘₐₓ of about 45 to about 58 μg per mL, and wherein said individual is known to have, or is suspected of having, AD. In a more specific embodiment, said plasma Cₘₐₓ is from about 47 to about 56 μg per mL. In a more specific embodiment, said plasma Cₘₐₓ is from about 48 to about 55 μg per mL. In a specific, preferred embodiment, said individual has mild to moderate AD. In another specific, preferred embodiment, said individual has MCI.

In another embodiment, the time to achieve plasma Cₘₐₓ will depend upon the individual to be treated, but is preferably between 0.70 to 3.00 hours. In various preferred embodiments, the tₘₐₓ (time to Cₘₐₓ) is from about 1.0 to 2.5 hours, or is from about 1.25 hour to about 2 hours, or is from about 1.40 to about 1.75 hours. Preferably, the t_{1/2} (half-life) is from about 6.00 to about 10.0 hours; from about 6.5 to about 9.5 hours; and from about 7 to about 9 hours. Preferably the AUC (area under the curve; total drug exposure) is from about 350 (hr*ug/mL) to 750 (hr*ug/mL); is from about 400 (hr*ug/mL) to 650 (hr*ug/mL); or is from about 450 (hr*ug/mL) to 700 (hr*ug/mL). In a specific, preferred embodiment, said individual has mild to moderate AD. In another specific, preferred embodiment, said individual has MCI.

In yet another embodiment, the time to achieve plasma Cₘₐₓ will depend upon the individual to be treated, but is preferably between 0.25 to 2.00 hours. In various preferred embodiments, the tₘₐₓ (time to Cₘₐₓ) is from about 0.25 to 1.75 hours, or is from about 0.50 hour to about 1.75 hours, or is from about 0.5 to about 1.25 hours. Preferably, the t_{1/2} (half-life) is from about 3.5 to about 8.5 hours; more preferably from about 4.0 to about 8.0 hours; and more preferably from about 4.8 to about 7.5 hours. Preferably the AUC (area under the curve; total drug exposure) is from about 250 (hr*ug/mL) to 700 (hr*ug/mL); is from about 300 (hr*ug/mL) to 650 (hr*ug/mL); or is from about 350 (hr*ug/mL) to 600 (hr*ug/mL). In a specific, preferred embodiment, said individual has mild to moderate AD. In another specific, preferred embodiment, said individual has MCI.

An AD diagnosis can be made using any known method. Typically, AD is diagnosed using a combination of clinical and pathological assessments. For example, progression or severity of AD can be determined using Mini Mental State Examination (MMSE) as described by Mohs et al. Int Psychogeriatr 8:195-203 (1996); Alzheimer's Disease Assessment Scale-cognitive component (ADAS-cog) as described by Galasko et al. Alzheimer Dis Assoc Disord, 11 suppl 2:S33-9 (1997); the Alzheimer's Disease Cooperative Study Activities of Daily Living scale (ADCS-ADL) as described by McKhann et al. Neurology 34:939-944 (1984); and the NINCDS-ADRDA criteria as described by Folstein et al. J. Psychiatr. Res. 12:189-198 (1975). In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993).

Individuals of any age may be treated by the methods of the invention, with the pharmaceutical compositions of the invention; however, the invention encompasses a preferred embodiment for treating or preventing Alzheimer's disease in individuals between the ages of 55 and 80. In various embodiments, individuals treated by the therapeutic or prophylactic methods of the invention may be from 55 to 70 years of age, 60 to 80 years of age, 55 to 65 years of age, 60 to 75 years of age, 65 to 80 years of age, 55 to 60 years of age, 60 to 65 years of age, 65 to 70 years of age, 70 to 75 years of age, 75 to 80 years of age, or 80 years old and older.

Thus, in one embodiment, the invention provides a method of treating an individual known or suspected of having Alzheimer's disease comprising administering an effective amount of R-flurbiprofen. In a specific embodiment, said individual is diagnosed as having mild to moderate Alzheimer's disease. In a more specific embodiment, said individual is diagnosed by a cognitive test as having mild to moderate AD. In a more specific embodiment, said cognitive test is the Mini-Mental State Exam (MMSE). In an even more specific embodiment, said individual has a score in said MMSE of from 26 to 19, inclusive. In another more specific embodiment, said individual has a score in said MMSE of from 18 to 10, inclusive. In another specific embodiment, said individual has a score in said MMSE of 26 to 10, inclusive.

R—NSAID Statin Conjugates

The invention provides a compound having one or more statins conjugated to one or more NSAIDs. The statin can be directly covalently conjugated to the NSAID or may be attached through a linker group. The conjugates of the invention have a general structure of X—SP-L-SP-L-SP—Y where X is one or more statin(s) or HMG-CoA reductase inhibitor(s), each SP is a spacer that is independent of one another and may or may not be present, each L is a linker that is independent of one another and may or may not be present, and Y is one or more NSAID(s). Desirably, the statin is atorvastatin, simvastatin, lovastatin, fluvastatin, pravastatin, cerivastatin, rosuvastatin, or pitavastatin, and the NSAID is flurbiprofen or R-flurbiprofen. In another aspect of the invention, the statin is conjugated to an Aβ₄₂ lowering agent.

Some examples of conjugates of the present invention include, but are not limited to the following structures. The clear wedge bonds at stereo centers indicate the bond is going behind the plane whereas the solid wedge bonds represent bond in front of the plane of paper. The stereochemistry of these molecules is known to the skilled artisan. 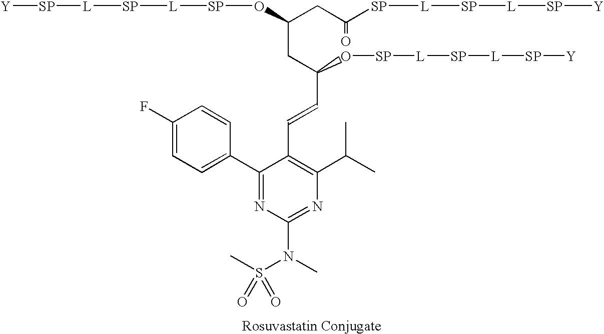
The above structure is a generic formula for a rosuvastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a rosuvastatin R-flurbiprofen conjugate is shown below. 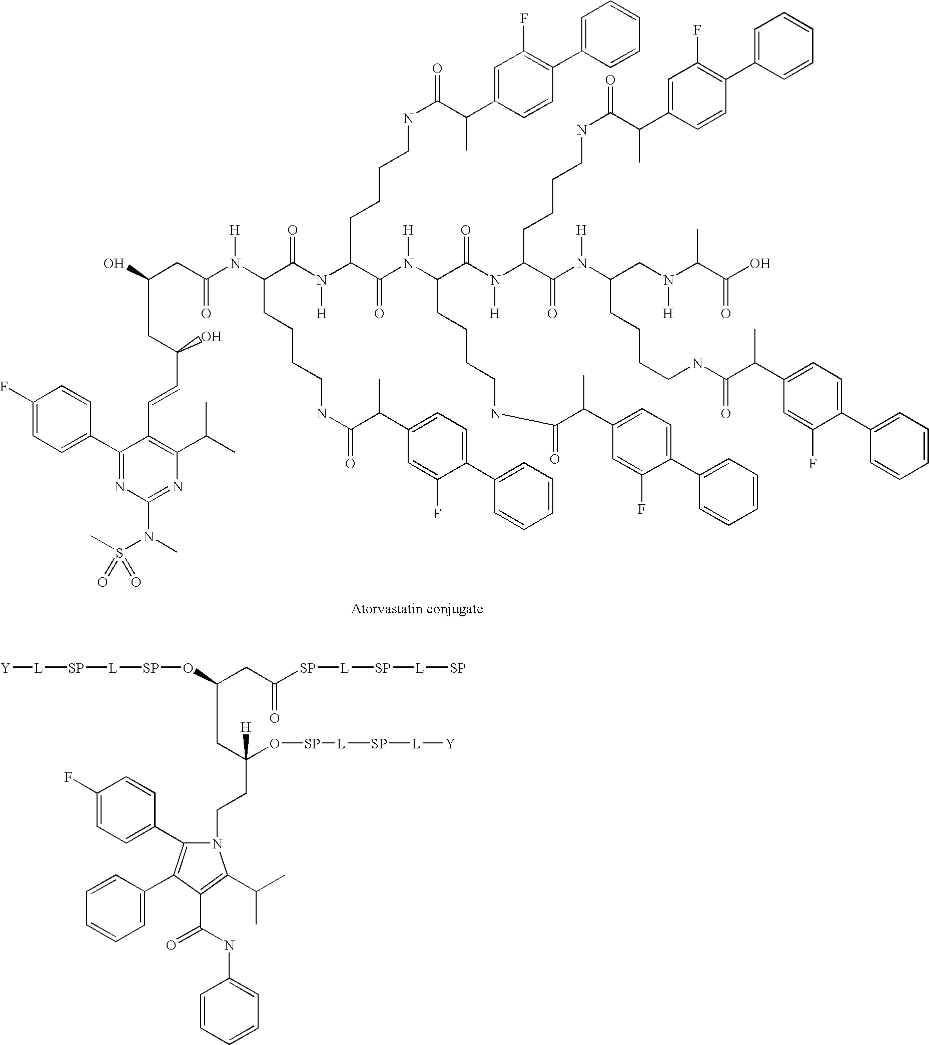
The above structure is a generic formula for a atorvastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of an atorvastatin R-flurbiprofen conjugate is shown below. 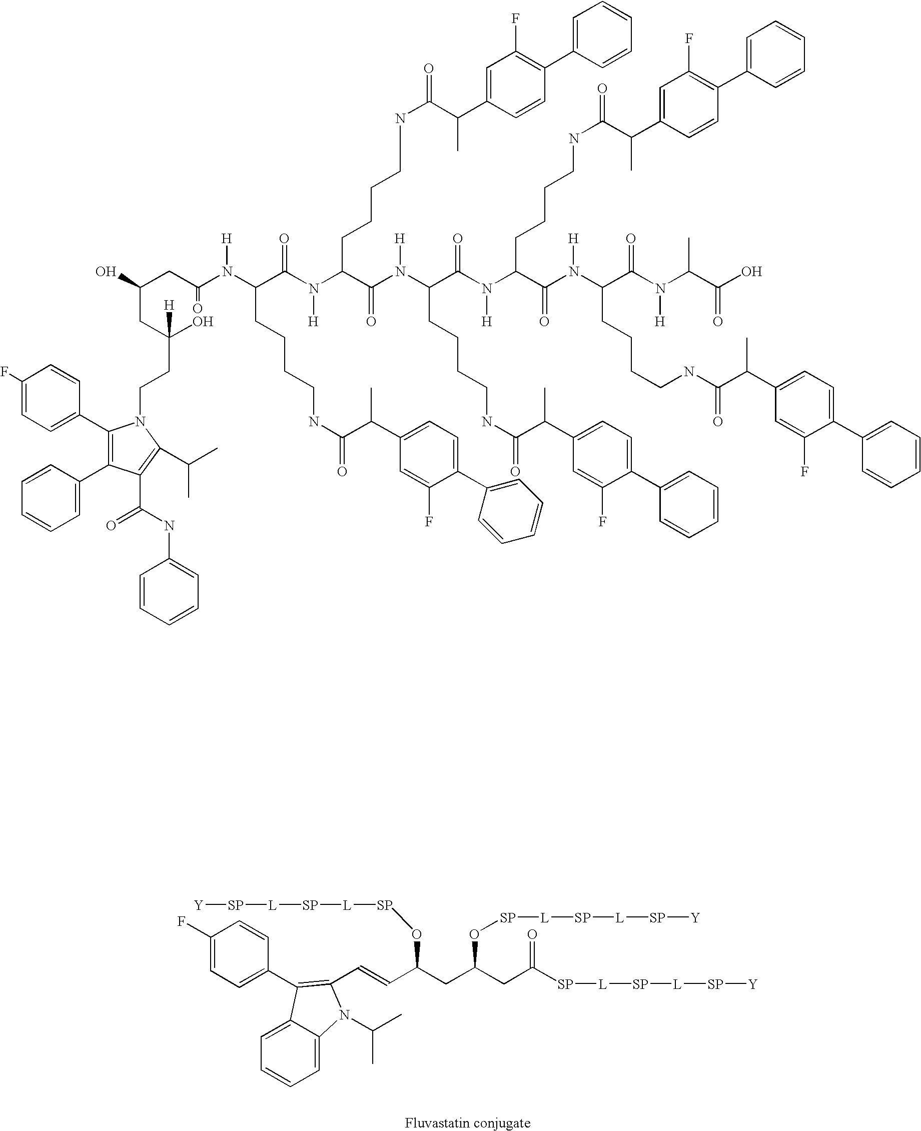
The above structure is a generic formula for a fluvastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a fluvastatin R-flurbiprofen conjugate is shown below. 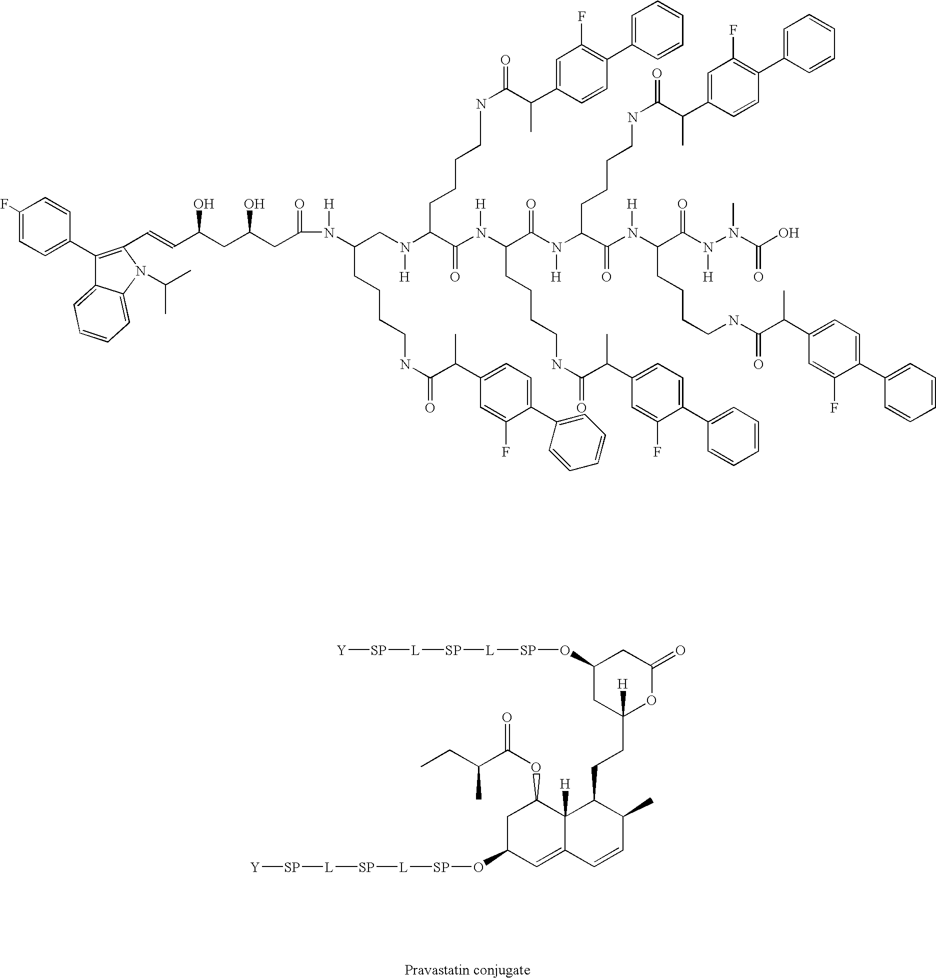
The above structure is a generic formula for a pravastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a pravastatin R-flurbiprofen conjugate is shown below. According to the invention, statins in the lactone form can be hydrolyzed, and linked through the resulting carboxylic acid moiety. 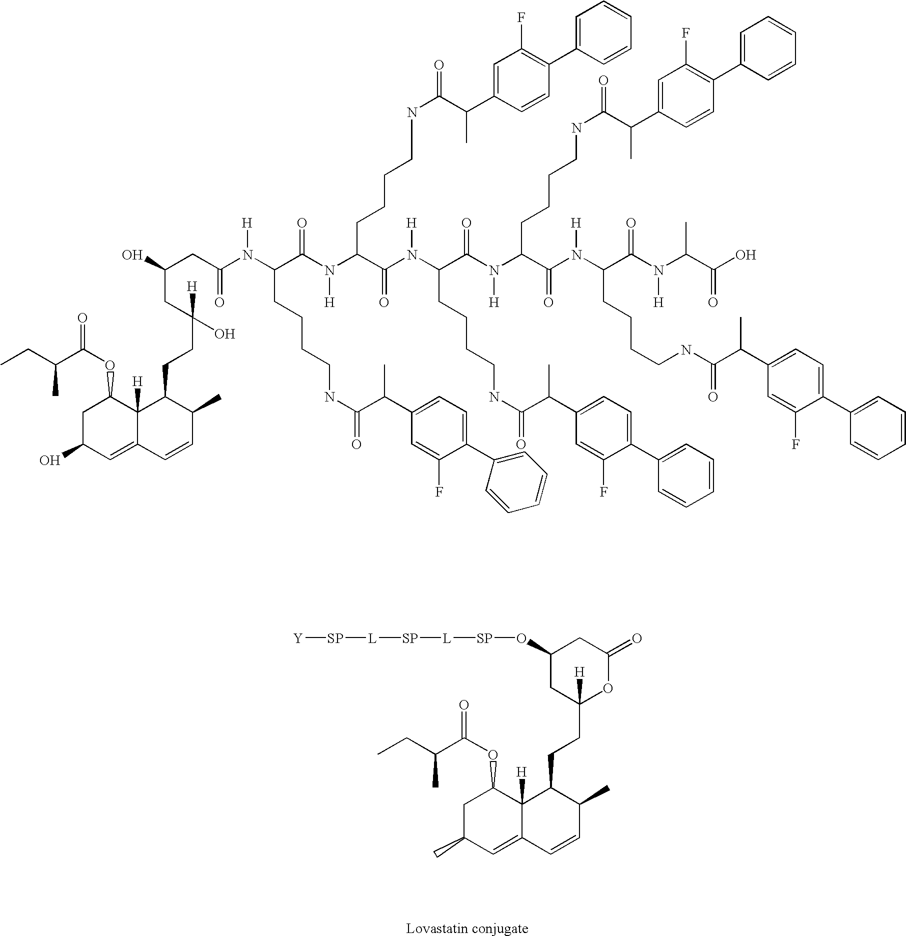
The above structure is a generic formula for a lovastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a lovastatin R-flurbiprofen conjugate is shown below. According to the invention, statins in the lactone form can be hydrolyzed, and linked through the resulting carboxylic acid moiety. 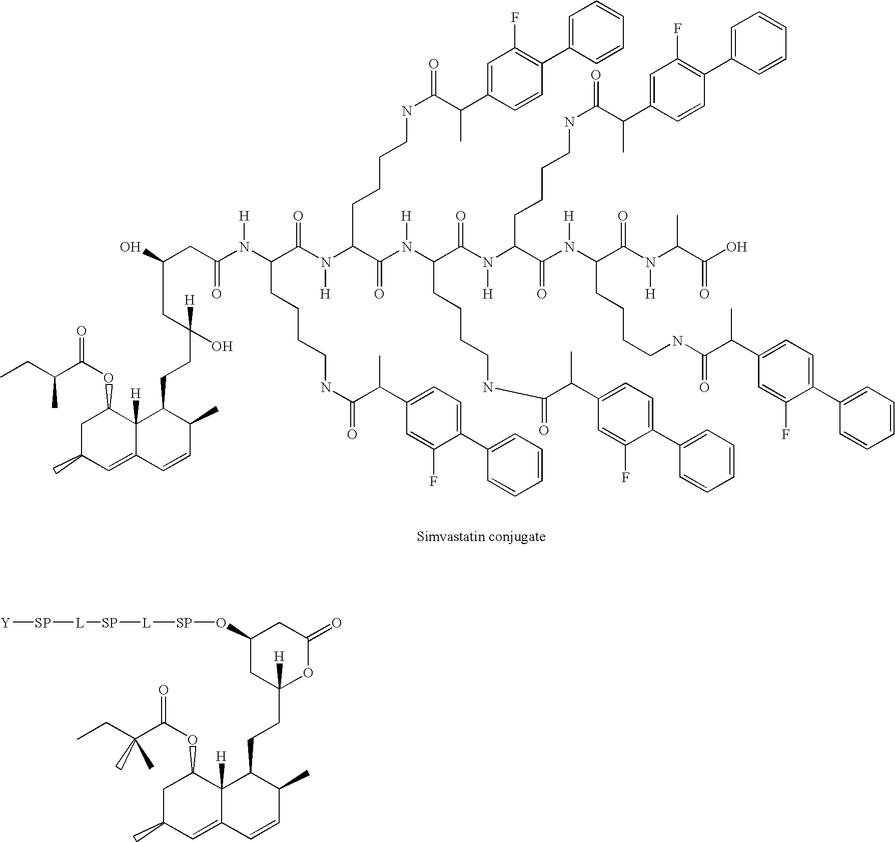
The above structure is a generic formula for a simvastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a simvastatin R-flurbiprofen conjugate is shown below. According to the invention, statins in the lactone form can be hydrolyzed, and linked through the resulting carboxylic acid moiety. 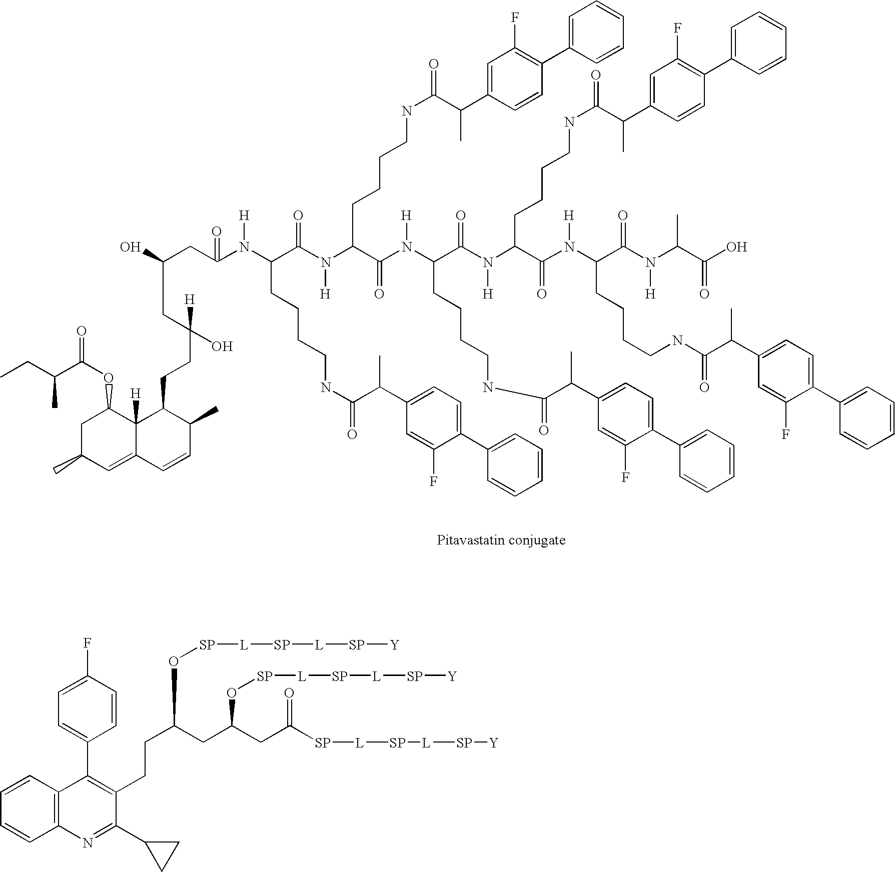
The above structure is a generic formula for a pitavastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a pitavastatin R-flurbiprofen conjugate is shown below. 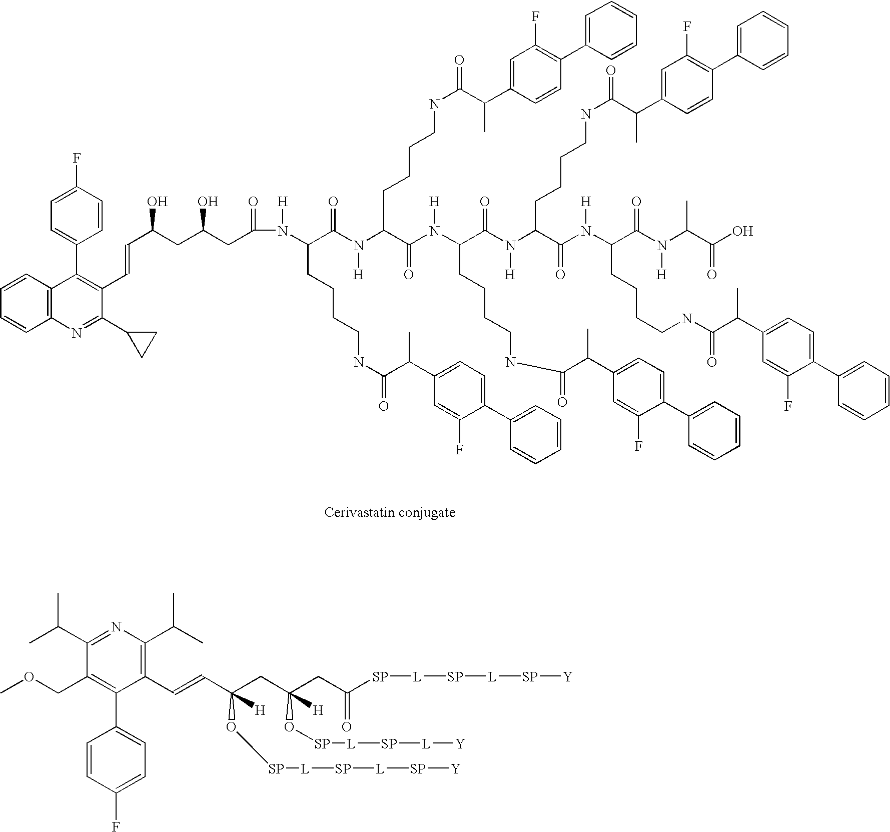
The above structure is a generic formula for a cerivastatin conjugate of the invention where each SP is a spacer that is independent of one another and may or may not be present, each L is a linker that is independent of one another and may or may not be present, and each Y independently represents one or more NSAIDs, R—NSAIDs, Aβ42 lowering agent, or other therapeutic agent such as dapsone, which may or may not be present. An example of a cerivastatin R-flurbiprofen conjugate is shown below. 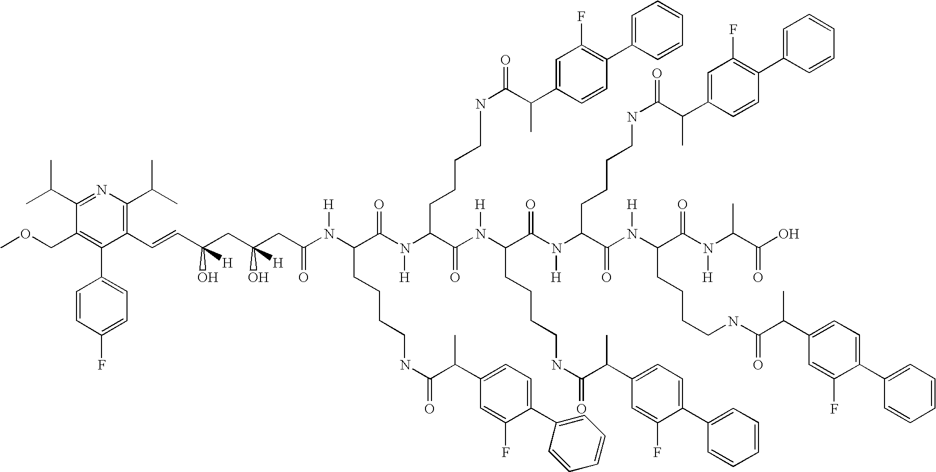
An example of a statin dapsone conjugate is shown below. The statin can be coupled through an amide linkage using the carboxylic acid moiety of the statin (for those which exist in the lactone form the lactone can be hydrolyzed to provide the carboxylic acid moiety using know procedures).

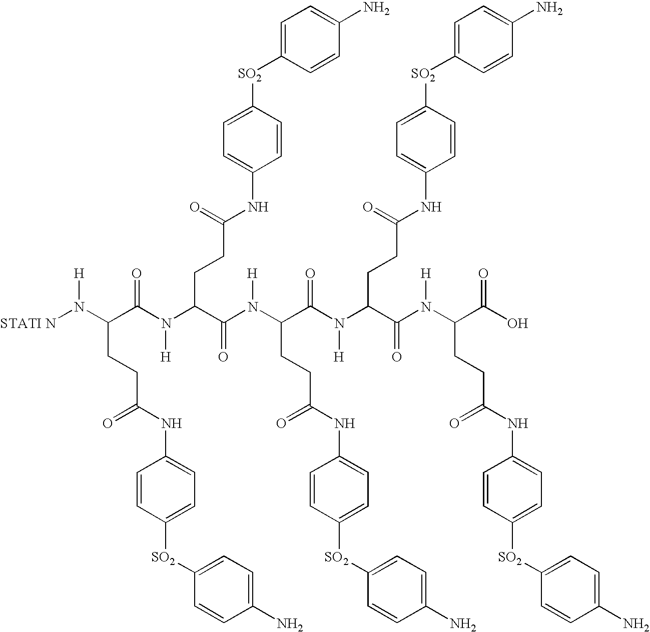

The weight to weight ratio of R—NSAID (or NSAID, Aβ42 lowering agent, or other therapeutic agent, i.e., dapsone) to statin in the conjugates of the invention can range from about 1 milligram (mg) NSAID to 10 mg statin to about 1 mg statin to about 50 mg NSAID. In a preferred aspect of this embodiment, a conjugate is provided having from about 1 mg to about 2000 mg R-flurbiprofen (or flurbiprofen), and from about 1 mg to about 200 mg statin selected from the group consisting of atorvastatin, simvastatin, lovastatin, fluvastatin, pravastatin, cerivastatin, rosuvastatin, and pitavastatin. In another preferred aspect of this embodiment, a conjugate is provided having from about 1 mg to about 2000 mg R-ibuprofen (or ibuprofen), and from about 1 mg to about 200 mg statin selected from the group consisting of atorvastatin, simvastatin, lovastatin, fluvastatin, pravastatin, cerivastatin, rosuvastatin, and pitavastatin. In yet another preferred aspect of this embodiment, a conjugate is provided having from about 1 mg to about 2000 mg lumiracoxib, and from about 1 mg to about 200 mg statin selected from the group consisting of atorvastatin, simvastatin, lovastatin, fluvastatin, pravastatin, cerivastatin, rosuvastatin, and pitavastatin. In another preferred aspect of this embodiment, a conjugate is provided having from about 1 mg to about 2000 mg dapsone and from about 1 mg to about 200 mg statin selected from the group consisting of atorvastatin, simvastatin, lovastatin, fluvastatin, pravastatin, cerivastatin, rosuvastatin, and pitavastatin. In another preferred aspect of this embodiment, a conjugate is provided having from about 1 mg to about 2000 mg sulindac, and from about 1 mg to about 200 mg statin selected from the group consisting of atorvastatin, simvastatin, lovastatin, fluvastatin, pravastatin, cerivastatin, rosuvastatin, and pitavastatin. In another preferred aspect of this embodiment, a conjugate is provided having from about 1 mg to about 2000 mg meclofenamic acid, and from about 1 mg to about 200 mg statin selected from the group consisting of atorvastatin, simvastatin, lovastatin, fluvastatin, pravastatin, cerivastatin, rosuvastatin, and pitavastatin. In another preferred aspect of this embodiment, a conjugate is provided having from about 1 mg to about 2000 mg of diclofenac, and from about 1 mg to about 200 mg of statin selected from the group consisting of atorvastatin, simvastatin, lovastatin, fluvastatin, pravastatin, cerivastatin, rosuvastatin, and pitavastatin. In one aspect of the invention, the conjugates of the invention have a ratio of NSAID to statin that is effective for treating Alzheimer's disease. In another aspect of the invention, the conjugates of the invention have a ratio of NSAID to statin that is effective for prophylaxis, slowing the progression of, and/or delaying the onset of symptoms of Alzheimer's disease.

TABLE 1
Weight/Weight Ratio of NSAID to Statin in Conjugates

| | | Range of Ratios |
|---|---|---|
| NSAID | Statin | of NSAID:Statin |
| R-flurbiprofen | Atorvastatin | 50:1 to 1:50 |
| R-flurbiprofen | Simvastatin | 50:1 to 1:50 |
| R-flurbiprofen | Lovastatin | 50:1 to 1:50 |
| R-flurbiprofen | Fluvastatin | 50:1 to 1:50 |
| R-flurbiprofen | Pravastatin | 50:1 to 1:50 |
| R-flurbiprofen | Cerivastatin | 50:1 to 1:50 |
| R-flurbiprofen | Rosuvastatin | 50:1 to 1:50 |
| R-flurbiprofen | Pitavastatin | 50:1 to 1:50 |
| Ibuprofen | Atorvastatin | 50:1 to 1:50 |
| Ibuprofen | Simvastatin | 50:1 to 1:50 |
| Ibuprofen | Lovastatin | 50:1 to 1:50 |
| Ibuprofen | Fluvastatin | 50:1 to 1:50 |
| Ibuprofen | Pravastatin | 50:1 to 1:50 |
| Ibuprofen | Cerivastatin | 50:1 to 1:50 |
| Ibuprofen | Rosuvastatin | 50:1 to 1:50 |
| Ibuprofen | Pitavastatin | 50:1 to 1:50 |
| Lumiracoxib | Atorvastatin | 50:1 to 1:50 |
| Lumiracoxib | Simvastatin | 50:1 to 1:50 |
| Lumiracoxib | Lovastatin | 50:1 to 1:50 |
| Lumiracoxib | Fluvastatin | 50:1 to 1:50 |
| Lumiracoxib | Pravastatin | 50:1 to 1:50 |
| Lumiracoxib | Cerivastatin | 50:1 to 1:50 |
| Lumiracoxib | Rosuvastatin | 50:1 to 1:50 |
| Lumiracoxib | Pitavastatin | 50:1 to 1:50 |
| Sulindac | Atorvastatin | 50:1 to 1:50 |
| Sulindac | Simvastatin | 50:1 to 1:50 |
| Sulindac | Lovastatin | 50:1 to 1:50 |
| Sulindac | Fluvastatin | 50:1 to 1:50 |
| Sulindac | Pravastatin | 50:1 to 1:50 |
| Sulindac | Cerivastatin | 50:1 to 1:50 |
| Sulindac | Rosuvastatin | 50:1 to 1:50 |
| Sulindac | Pitavastatin | 50:1 to 1:50 |
| Meclofenamic acid | Atorvastatin | 50:1 to 1:50 |
| Meclofenamic acid | Simvastatin | 50:1 to 1:50 |
| Meclofenamic acid | Lovastatin | 50:1 to 1:50 |
| Meclofenamic acid | Fluvastatin | 50:1 to 1:50 |
| Meclofenamic acid | Pravastatin | 50:1 to 1:50 |
| Meclofenamic acid | Cerivastatin | 50:1 to 1:50 |
| Meclofenamic acid | Rosuvastatin | 50:1 to 1:50 |
| Meclofenamic acid | Pitavastatin | 50:1 to 1:50 |
| Diclofenac | Atorvastatin | 50:1 to 1:50 |
| Diclofenac | Simvastatin | 50:1 to 1:50 |
| Diclofenac | Lovastatin | 50:1 to 1:50 |
| Diclofenac | Fluvastatin | 50:1 to 1:50 |
| Diclofenac | Pravastatin | 50:1 to 1:50 |
| Diclofenac | Cerivastatin | 50:1 to 1:50 |
| Diclofenac | Rosuvastatin | 50:1 to 1:50 |
| Diclofenac | Pitavastatin | 50:1 to 1:50 |

Linkers

According the present invention, a combination of R—NSAID and statin, NSAID and statin, Aβ42 lowering agent and statin, COX inhibitor and statin, anti-inflammatory agent and statin can be conjugated to a polymer. Preferably the polymer is a water soluble polymer. The specific polymer used to conjugate the two active agents can be selected based on the chemical nature of the two active agents. The polymer can be conjugated to any reactive moiety on the active agents as long as the active agents can be liberated from the linker without destroying the therapeutic properties of the active agents. Any known linking reaction can be used to conjugate the NSAIDs and statins to the linker moiety.

Polyglutamic acid (PG) is one polymer that offers several advantages in the present invention. First, it contains a large number of side chain carboxyl functional groups for drug attachment. Second, PG can be readily degraded by lysosomal enzymes to its nontoxic basic component, l-glutamic acid, d-glutamic acid and dl-glutamic acid. The polyglutamic acids (or polyaspartic acids) can be derivatized using standard chemical procedures at the carboxylic moiety (e.g., with a compound having two free amino groups) to provide a method of linker a carboxylic acid functional group of a statin or NSAID to the derivatized polyglutamic acid (or polyapartic acids).

Preferred polymers include, but are not limited to poly(l-glutamic acid), poly(d-glutamic acid), poly(dl-glutamic acid), poly(l-aspartic acid), poly(d-aspartic acid), poly(dl-aspartic acid), poly(l-lysine), poly(d-lysine), poly(dl-lysine), copolymers of the above listed polyamino acids with polyethylene glycol, polycaprolactone, polyglycolic acid and polylactic acid, as well as poly(2-hydroxyethyl 1-glutamine), chitosan, carboxymethyl dextran, hyaluronic acid, human serum albumin and alginic acid.

The polymers that can be used in the conjugates of the invention can range in molecular weights of about 200 to 300 daltons up to about 100,000 daltons. Preferably, the polymers for use in the conjugates of the invention have a lower end molecular weight of about 200, about 300, about 500, about 1,000, about 2,000, about 3,000, about 4,000, about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21 ,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000, about 41,000, about 42,000, about 43,000, about 44,000, about 45,000, about 46,000, about 47,000, about 48,000, about 49,000, to about 50,000 daltons. Preferably, the polymers for use in the conjugates of the invention have a higher end molecular weight of about 51,000, about 52,000, about 53,000, about 54,000, about 55,000, about 56,000, about 57,000, about 58,000, about 59,000, about 60,000, about 61,000, about 62,000, about 63,000, about 64,000, about 65,000, about 66,000, about 67,000, about 68,000, about 69,000, about 70,000, about 71,000, about 72,000, about 73,000, about 74,000, about 75,000, about 76,000, about 77,000, about 78,000, about 79,000, about 80,000, about 81,000, about 82,000, about 83,000, about 84,000, about 85,000, about 86,000, about 87,000, about 88,000, about 89,000, about 90,000, about 91,000, about 92,000, about 93,000, about 94,000, about 95,000, about 96,000, about 97,000, about 98,000, about 99,000, to about 100,000 daltons.

In yet another aspect of the invention, the amount of statin and R—NSAID (or COX-2 selective compound or Aβ42 lowering agent or other agent, i.e., dapsone) conjugated per water soluble polymer can vary. For example, the lower amount of therapeutic agent in the conjugate is from about 1%, about 2%, about 3%, about 4%, about 5%, about 6%, about 7%, about 8%, about 9%, or about 10%, about 11%, about 12%, about 13%, about 14%, about 15%, about 16%, about 17%, about 18%, about 19%, about 20%, about 21% about 22%, about 23%, about 24%, to about 25% (w/w) therapeutic agent relative to the mass of the conjugate. The upper amount of therapeutic agent(i.e., NSAID and statin) in the conjugate is from about 26%, about 27%, about 28%, about 29%, about 30%, about 31% about 32%, about 33%, about 34%, about 35%, about 36%, about 37%, about 38%, about 39%, about 40%, about 45%, about 50%, to about 60% or more (w/w) of therapeutic agent relative to the mass of the conjugate.

In another aspect of the invention, the number of molecules of statin conjugated per molecule of water soluble polymer can vary. At the lower end, such a composition may comprise from about 1, about 2, about 3, about 4, about 5, about 6, about 7, about 8, about 9, about 10, about 11, about 12, about 13, about 14, about 15, about 16, about 17, about 18, about 19, to about 20 or more molecules of statin per molecule of water soluble polymer. For the upper range, the conjugate can comprise from about 21, about 22, about 23, about 24, about 25, about 26, about 27, about 28, about, 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 about 61, about 62, about 63, about 64, about 65, about 66, about 67, about 68, about 69, about 70, about 71, about 72, about 73, about 74, to about 75 or more molecules or more of statin molecules per molecule of water soluble polymer.

In another aspect of the invention, the number of molecules of R—NSAID (or COX-2 selective compound or Aβ42 lowering agent or other agent, i.e., dapsone) conjugated per molecule of water soluble polymer can vary. At the lower end, such a composition may comprise from about 1, about 2, about 3, about 4, about 5, about 6, about 7, about 8, about 9, about 10, about 11, about 12, about 13, about 14, about 15, about 16, about 17, about 18, about 19, to about 20 or more molecules of R—NSAID (or COX-2 selective compound or Aβ42 lowering agent or other agent, i.e., dapsone) per molecule of water soluble polymer. At the higher end, such a composition may comprise from about 21, about 22, about 23, about 24, about 25, about 26, about 27, about 28, about, 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 about 61, about 62, about 63, about 64, about 65, about 66, about 67, about 68, about 69, about 70, about 71, about 72, about 73, about 74, to about 75 or more molecules or more of R—NSAID (or NSAID, COX-2 selective compound, Aβ42 lowering agent or other agent, i.e., dapsone) molecules per molecule of water soluble polymer.

A variety of other known linker agents for conjugate therapeutic agents can be used in the conjugates of the invention. These linker agents include, but are not limited to, polyethylene glycols (PEG), carbohydrates, sugars, and proteins. Examples of U.S. patents (all of which are incorporated by reference in their entireties) that describe formulations suitable for use as linking agents include, but are not limited to, U.S. Pat. No.5,356,630; U.S. Pat. No. 5,797,898 U.S. Pat. No.5,874,064; U.S. Pat. No. 5,548,035; U.S. Pat. No.5,532,287; U.S. Pat. No.5,284,831; U.S. Pat. No.5,525,727; U.S. Pat. No. 5,741,329; U.S. Pat. No.5,820,883; U.S. Pat. No. 5,955,068; U.S. Pat. No. 6,001,395; U.S. Pat. No.6,013,853; U.S. Pat. No. 6,060,582; U.S. Pat. No.6,113,943; U.S. Pat. No. 6,123,861; U.S. Pat. No.6,060,082; U.S. Pat. No. 6,041,253; U.S. Pat. No. 6,018,678; U.S. Pat. No.6,007,845; U.S. Pat. No.6,004,534; U.S. Pat. No.6,002,961; U.S. Pat. No. 5,985,309; U.S. Pat. No.5,947,921; U.S. Pat. No.5,912,017; U.S. Pat. No.5,911,223; U.S. Pat. No.5,874,064; U.S. Pat. No.5,855,913; U.S. Pat. No.5,846,565; U.S. Pat. No. 5,837,752; U.S. Pat. No. 5,814,599; U.S. Pat. No. 5,804,178; U.S. Pat. No. 5,797,898; U.S. Pat. No.5,770,417; U.S. U.S. Pat. No.5,770,193; U.S. Pat. No.5,762,904; U.S. Pat. No.5,759,830; U.S. Pat. No.5,749,847; U.S. Pat. No.5,736,372; U.S. Pat. No. 5,718,921; U.S. Pat. No. 5,696,175; U.S. Pat. No. 5,667,491; U.S. Pat. No.5,654,381; U.S. Pat. No.5,651,986; U.S. Pat. No.5,629,009; U.S. Pat. No.5,626,862; U.S. Pat. No. 5,593,974; U.S. Pat. No. 5,578,325; U.S. Pat. No. 5,562,099; U.S. Pat. No. 5,545,409; U.S. Pat. No.5,543,158; U.S. Pat. No.5,514,378; U.S. Pat. No.5,512,600; U.S. Pat. No. 5,500,161; U.S. Pat. No. 5,487,390; U.S. Pat. No. 5,399,665; U.S. Pat. No. 5,356,630; U.S. Pat. No.5,330,768; U.S. Pat. No.5,286,763; U.S. Pat. No.5,149,543; U.S. Pat. No. 5,128,420; U.S. Pat. No. 5,122,367; U.S. Pat. No. 5,100,668; U.S. Pat. No.5,019,379; U.S. Pat. No.5,010,167; U.S. Pat. No.4,948,587; U.S. Pat. No.4,946,929; U.S. Pat. No. 4,933,431; U.S. Pat. No. 4,933,185; U.S. Pat. No. 4,921,757; U.S. Pat. No.4,916,204; U.S. Pat. No.4,906,474; U.S. Pat. No.4,900,; U.S. Pat. No.4,898,734; U.S. Pat. No. 4,891,225; U.S. Pat. No. 4,888,176; U.S. Pat. No. 4,886,870; U.S. Pat. No.4,863,735; U.S. Pat. No.4,863,611; U.S. Pat. No.4,861,627; U.S. Pat. No.4,857,311; U.S. Pat. No. 4,846,786; U.S. Pat. No. 4,806,621; U.S. Pat. No. 4,789,724; U.S. Pat. No.4,780,212; U.S. Pat. No.4,779,806; U.S. Pat. No.4,767,402; U.S. Pat. No.4,757,128; U.S. Pat. No. 4,657,543; U.S. Pat. No.4,638,045; and U.S. Pat. No.4,591,496.

The above referenced patents demonstrate how the various variables associated with the conjugates of the present invention can be independently varied to more particularly define specific classes of conjugates encompassed by this invention. It is understood to the skilled artisan that a variety of linkers can be used in the conjugates of the invention and such conjugates can be made according to techniques available to the ordinary skilled artisan, i.e., like those synthetic techniques described in the above-referenced patents.

Spacer

The statin R—NSAID (or COX-2 selective compound or Aβ42 lowering agent or other agent, i.e., dapsone) conjugates of the present invention can comprise an intermediate self-immolative spacer moiety which spaces and covalently links together the therapeutic moieties (R—NSAID and statin) and the linkers. As used herein “a self-immolative spacer” is defined as a bifunctional chemical moiety which is capable of covalently linking together two spaced chemical moieties into a normally stable tripartate molecule or moiety, releasing one of said spaced chemical moieties from the tripartate molecule (or moiety) by means of enzymatic cleavage or spontaneous chemical cleavage. In accordance with the present invention, the self-immolative spacer is covalently linked at both of its ends to a linker so as to space and covalently link together therapeutic moieties into a tripartate molecule composed of a statin, a R—NSAID (or COX-2 selective compound or Aβ42 lowering agent or other agent, i.e., dapsone), and the linker/spacer moiety, providing a stable conjugate. In one aspect, the conjugate is pharmacologically inactive in the absence of the target enzyme capable of cleaving the linker but which is enzymatically cleavable by a target enzyme at the bond covalently linking the spacer moiety and the linker moiety to thereby effecting release of the linker moiety from the tripartate molecule. The cleavage, in turn, will activate the self-immolating character of the spacer moiety and initiate spontaneous cleavage of the bond covalently linking the spacer moiety to the linker or therapeutic moiety, thereby effecting release of the linker or therapeutic agent in a pharmacologically active form.

### 2.0. DEFINITIONS

As used herein, the term “preventing an increase in a symptom” or “delaying the progression” refers to both not allowing a symptom to increase or worsen, as well as reducing the rate of increase in the symptom. For example, a symptom can be measured as the amount of particular disease marker, i.e., a protein. Preventing an increase, according to the definition provided herein, means that the amount of the protein does not increase or that the rate at which it increases is reduced.

As used herein, the term “statin” refers to a class of pharmaceuticals known as 3-hydroxy-3-methylglutaryl-Coenzyme-A reductase inhibitors (“HMG-CoA inhibitor”), the rate-limiting enzyme that converts HMG-CoA into mevalonate. HMG-CoA reductase inhibitors include, but are not limited to, atorvastatin, simvastatin, lovastatin, fluvastatin, pravastatin, cerivastatin, rosuvastatin, pitavastatin, compounds described in WO 00/96311, WO 00/28981, WO 86/07054 ,U.S. Pat. No. 4,647,576, U.S. Pat. No. 4,686,237, all of which are hereby incorporated by reference in their entireties. Lescol (fluvastatin sodium) is commercially available from Novartis and is [R*,S*-(E)]-(.±.)-7-[3-(4-fluorophenyl)-1-(1-methylethyl)-1H-indo-1-2-yl]-3,5-dihydroxy-6-heptenoic acid, monosodium salt. Lipitor (atorvastatin calcium) is commercially available from Parke Davis and is [R-(R*,R*)]-2-(4-fluorophenyl)-β,δ-dihydroxy-5-(1-methylethyl)-3-phenyl-4-[(phenyl-amino)carbonyl]-1H-pyrrole-1-heptanoic acid, calcium salt (2:1) trihydrate. Mevacor (lovastatin) is commercially available from Merck and is [1S-[1-α-(R*),3-α,7β,8β(2S,4S),8aβ]]-1,2,3,7,8,8a-hexahydro-3,7-dimethyl-8-[2-(tetrahydro-4-hydroxy-6-oxo-2H -pyran-2-yl)ethyl]-1-naphthalenyl-2-menthylbutanotate. Pravachol (pravastatin sodium) is commercially available from Bristol-Myers Squibb and is [1S-[1α(βS*,εS*), 2α, 6α, 8α(R*),8aα]]-1,2,6,7,8,Sa-hexahydro-β, δ, 6-tri-hydroxy-2-methyl-8-(2-methyl-1-oxobutoxy)-1-naphthalene-heptanoic acid monosodium salt. Zocor (simvastatin) is commercially available from Merck and is butanoic acid, [1S-1α,3α,7β8β(2S*,4S*),-8aβ]]-2,2-dimethyl-1,2,3,7,8,8a-hexahydro-3,7-dimethyl-8-[2-(tetrahydro-4-hydroxy-6-oxo-2H-pyran-2-yl)-ethyl]-1-naphthalenyl ester.

As used herein, the term “Aβ₄₂ lowering” refers the capability to reduce the amount of Aβ₄₂ present and/or being produced (i.e., in transgenic mice overexpressing familial Alzheimer's disease genes and/or according to the cell culture based assays described herein). Levels of Aβ₄₂ can be determined with an ELISA assay configured to detect Aβ_{42.} Methods of determining Aβ₄₂ levels are described in the examples and references cited therein.

“A level of Aβ₄₂” in a human subject means the amount or concentration of Aβ₄₂ in at least one of the organs or tissues of the subject (e.g., brain or a body fluid such as CSF, plasma or blood). The brain level Aβ₄₂ can be measured by e.g., PET scan and other imaging techniques. The Aβ₄₂ level in CSF or plasma or blood can be measured by e.g., an ELISA assay using an antibody specific to the Aβ₄₂ peptide as described below.

As used herein, the term “treating Alzheimer's disease” refers to a slowing of or a reversal of the progress of the disease, or delaying the appearance or onset of one or more symptoms of the disease. Treating Alzheimer's disease includes reducing the symptoms of the disease.

As used herein, the term “preventing Alzheimer's disease” refers to a slowing or delaying of the onset of the disease.

As used herein, the terms “a polyglutamic acid” or “polyglutamic acids” include poly (l-glutamic acid), poly (d-glutamic acid) and poly (dl-glutamic acid). The terms “a polyaspartic acid” or “polyaspartic acids” include poly (l-aspartic acid), poly (d-aspartic acid), poly (dl-aspartic acid). The terms “apolylysine” or “polylysine” include poly (l-lysine), poly (d-lysine), poly (dl-lysine). The terms “a water soluble polyamino acid”, “water soluble polyamino acids”, or “water soluble polymer of amino acids” include, but are not limited to, polyglutamic acid, polyaspartic acid, polylysine, and amino acid chains comprising mixtures of glutamic acid, aspartic acid, and/or lysine. In certain embodiments, the terms “a water soluble polyamino acid”, “water soluble polyamino acids”, or “water soluble polymer of amino acids” include amino acid chains comprising combinations of glutamic acid and/or aspartic acid and/or lysine, of either d and/or l isomer conformation. In certain preferred embodiments, such a “water soluble polyamino acid” contains one or more glutamic acid, aspartic acid, and/or lysine residues. “Water soluble polyamino acids” can also comprise any natural, modified, or unusual amino acid as are known to the skilled artisan. In certain embodiments, a water soluble polymer of amino acids that contains more than one different type of amino acid residue is sometimes referred to herein as a “co-polymer”.

As used herein, the terms “polypeptide,” “protein,” and “peptide” interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

### 3.0. ASSAYS FOR COX-½ ACTIVITY AND Aβ LEVELS

In vitro cellular COX inhibition can be determined using specific assays for inhibition of COX-1 and COX-2. An art-known cellular assay for determining COX inhibition is based on the production of prostaglandin-E₂ from exogenous arachidonic acid in cells expressing COX-1, COX-2, or a combination thereof. COX enzymes (prostaglandin H synthase) catalyze the rate-limiting step in prostaglandin synthesis from arachidonic acid. Cell lines are known and available that express at least one form of the enzyme. For example, a human skin fibroblast line can be induced with IL-1 to synthesize COX-2, and a kidney epithelial cell line 293 has been stably transfected to constitutively express COX-1. In these assays, arachidonic acid can be added exogenously to increase signal to readably detectable levels. Thus, the amount of prostaglandin-E₂ in the extracellular medium can be assayed by radioimmunoassay, for measuring COX activity. IC₅₀ values for compounds for COX-1 and COX-2 can be determined by an ordinary skilled artisan. Anti-inflammatory activities of compounds can be determined using the art-known rat paw edema assay.

The effects of the compositions and compounds of the invention can be determined by examining the secretion of Aβ₄₂ by a CHO cell line that expresses APP. Untreated cell cultures, cell cultures treated with a compound of the invention and a carrier, carrier treated cell cultures can be examined and compared, and Aβ₄₂ levels secretion levels can be determined.

A CHO (Chinese hamster ovary) cell line expressing APP can be culture for an appropriate amount of time and the supernatants analyzed for Aβ₄₂ and Aβ₄₀ levels using end-specific Aβ₄₂ and Aβ₄₀ELISAs (Suzuki et al. (1994) Science 264:336-340). Supernatants from cell cultures grown in the presence of varying concentrations of the compositions of the invention and active ingredients thereof, ranging from about 0.1 μM to about 500 μM are analyzed for Aβ₄₂ and Aβ₄₀ levels. Supernatants from control cell cultures treated with carrier and receiving no treatment are also analyzed for Aβ₄₂ and Aβ₄₀ levels. Compounds and compositions which alter Aβ₄₂ levels by more than about 15% as compared to the cultures grown in the presence of carrier under similar conditions are said to lower Aβ₄₂ levels.

For further description of assays, cell line, and techniques capable of assessing COX inhibitory activity and Aβ₄₂ lowering activity see, e.g., WO 01/78721, and references cited therein, all of which are incorporated herein in their entirety.

### 4.0. ADDITIONAL COMBINATION THERAPY

The invention further provides additional combination therapy strategies for treating neurodegenerative disorders such as Alzheimer's disease, MCI, and dementia. According to this aspect of the invention, an individual in need of treatment is administered an effective amount of an R—NSAID (e.g., R-flurbiprofen), at least one statin (such as atorvastatin, simvastatin, lovastatin, fluvastatin, pravastatin, cerivastatin, rosuvastatin, and pitavastatin) and at least one compound selected from the group consisting of NSAIDs, COX-2 inhibitors (cyclooxygenase-2), β-secretase inhibitors, and γ-secretase inhibitors, acetylcholine esterase inhibitors, and GABA-A alpha inverse agonist (see WO 00/27382, WO 96/25948, WO 98/50385 which are herein incorporated by reference in there entireties). Preferred acetylcholine esterase inhibitors include tacrine, donepezil, rivastigrnine, and galantamine. The combination therapy of the invention is thought to provide a synergistic effect in reducing Aβ42 levels and is surprisingly thought to be especially effective for treating and preventing neurodegenerative disorders including Alzheimer's disease, dementia, and MCI. The invention further encompasses compositions comprising the combination of active ingredients of this aspect of the invention.

According to another aspect of the invention, an individual in need of such treatment is administered an effective amount of R-flurbiprofen, at least one statin such as atorvastatin, simvastatin, lovastatin, fluvastatin, pravastatin, cerivastatin, rosuvastatin, and pitavastatin, and at least one NSAD. According to a preferred aspect of this embodiment the NSAID is selected from the group consisting of 5,5-dimethyl-3-(3-fluorophenyl)-4-(4-methylsulfonyl)phenyl-2(5H)-furanone, 5,5-dimethyl-3-isopropyloxy-4-(4′-methylsulfonylphenyl)-2(5H)-furanone, resveratrol, flufemic acid, meclofenamic acid, fenoprofen, carprofen, ibuprofen, ketoprofen, sulindac, indomethacin, naproxen, etolodac, tiaprofenic, suprofen, ketorolac, pirprofen, indoprofen, benoxaprofen, oxaprozin, diflunisal, and nabumetone.

The treatment regime used in the combination therapy can involve administration of a composition comprising the combination of active ingredients, the concomitant administration of separate compositions, each comprising at least one active ingredient. Furthermore, the administration of the active ingredients can be performed at different times and/or different routes. For example, a composition having one active ingredient can be administered in the morning, and a composition having the other active ingredients can be administered in the evening. Another example would involve the administration of a composition having two active ingredients orally while the third active ingredient is administered intravenously.

### 5.0. PREPARATION OF COMPOUNDS OF THE INVENTION

The compounds of the invention can be prepared by a variety of art known procedures. In one aspect, the R—NSAID employed in the compositions and methods disclosed herein can be selected from the group consisting of selected R-flurbiprofen, R-ibuprofen, R-ketoprofen, R-naproxen, R-tiaprofenic acid, R-suprofen, R-carprofen, R-pirprofen, R-indoprofen, and R-benoxaprofen. The R—NSAID can also be a cyclized derivative of an arylpropionic acid, such as R-ketorolac, or an arylacetic acid, such as R-etodolac. Descriptions of specific NSAIDs and their preparation can be found in various publications. R-Ibuprofen is described in U.S. Pat. No. 6,255,347. Ketoprofen is described in U.S. Pat. No. 3,641,127. Flurbiprofen is described in U.S. Pat. No. 3,755,427. Ketorolac is described in U.S. Pat. No. 4,089,969.

A large number of the NSAIDs useful according to the invention are commercially available either in the form of racemic mixtures or as optically pure enantiomers. In all cases racemic mixtures contain equal amounts of the R- and S-isomers of the NSAID are provided. For example, the following racemates can be obtained through Sigma Chemical Co.: ketoprofen, flurbiprofen, etodolac, suprofen, carprofen, indoprofen and benoxaprofen. Naproxen, marketed as the S-isomer only, is also available from this source. Additionally, many commercial sources exist for the stereospecific R-isomers of many NSAIDs. R-ketoprofen, R-flurbiprofen and R-ketorolac, for example, are available through Sepracor, Inc.; R-naproxen can be obtained as the sodium salt through Sigma Chemical Co.; R-etodolac is available from Wyeth-Ayerst; R-tiaprofenic acid is available through Roussel (France, Canada, Switzerland, Spain, Denmark, Italy); R-suprofen is manufactured by McNiel Pharmaceuticals; R-carprofen is available from Roche; R-pirprofen is available through Ciba (France, Belgium, Denmark); R-indoprofen can be obtained through Carlo Elba (Italy, U.K.); and R-benoxaprofen is manufactured by Eli Lilly Co. HMG-CoA inhibitors or statins for use in this invention are any selective, inhibitor of HMG-CoA reductase, the rate-limiting enzyme that converts HMG-CoA into mevalonate, including: lovastatin, marketed under the trademark MEVACOR by Merck, and described in U.S. Pat. No. 4,231,938; simvastatin, marketed under the trademark ZOCOR by Merck, and described in U.S. Pat. No. 4,444,784; pravastatin, marketed under the trademark PRAVACOL by Bristol-Myers-Squibb, and described in U.S. Pat. No. 4,346,227; atorvastatin calcium, marketed under the name LIPITOR by Parke-Davis, and described in U.S. Pat. No. 5,273,995; cerivastatin sodium, marketed under the name BAYCOL, by Bayer, and described in U.S. Pat. No. 5,177,080; fluvastatin sodium, marketed under the name LESCOL, by Novartis Pharmaceuticals, and described in U.S. Pat. No. 5,354,772; Rosuvastatin (Astrazeneca, U.S. Pat. No.5,260,440) and Pitavastatin (cas registry number CAS-147511-69-1). All of the patents referenced in this section are hereby incorporated by reference in their entireties.

The conjugates of the invention can be prepared by one of ordinary skill in the art of organic chemistry synthesis.

### 6.0. FORMULATION AND ROUTES OF DELIVERY

The compositions according to the invention are those suitable for enteral, such as oral or rectal, transdermal, topical, and parenteral administration to an individual, for the prevention and/or treatment of neurodegenerative disorders including Alzheimer's disease, dementia, and/or MCI. Such compositions can comprise an effective amount of a compound or compounds as described herein, alone or in combination, and with one or more pharmaceutically acceptable carriers.

In conjunction with another active ingredient, a compound of the invention may be administered either simultaneously, before or after the other active ingredient, either separately by the same or different route of administration or together in the same pharmaceutical formulation.

In the methods of the invention, the pharmacologically active compound(s) of the invention can be manufactured as a pharmaceutical composition comprising an effective amount of the compound(s) in conjunction or admixture with excipients or carriers suitable for either enteral or parenteral application. Preferred are tablets and gelatin capsules comprising the active ingredient together with a) diluents, e.g. lactose, dextrose, sucrose, mannitol, sorbitol, cellulose and/or glycine; b) lubricants, e.g., silica, talcum, stearic acid, its magnesium or calcium salt and/or polyethylene glycol; for tablets also c) binders e.g., magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient. Tablets may be either film coated or enteric coated according to methods known in the art.

Suitable formulations for transdermal application include an effective amount of a compound(s) of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

The compositions of the present invention can be prepared in any desired form, for example, tablets, powders, capsules, suspensions, solutions, elixirs, and aerosols. Carriers such as starches, sugars, microcrystalline cellulose, diluents, granulating agents, lubricants, binders, disintegrating agents, and the like may be used in the cases of oral solid preparations. Oral solid preparations (such as powders, capsules, and tablets) are preferred over oral liquid preparations. The most preferred oral solid preparations are tablets. If desired, tablets may be coated by standard aqueous or non-aqueous techniques.

In addition to the common dosage forms set out above, the compounds of the present invention may also be administered by controlled release means and/or delivery devices such as those described in U.S. Pat. Nos.: 3,845,770; 3,916,899; 3,536,809; 3,598,123; and 4,008,719, the disclosures of which are hereby incorporated by reference in their entireties.

Pharmaceutical compositions of the present invention suitable for oral administration may be presented as discrete units such as capsules, cachets, or tablets, or aerosol sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the conventional methods of pharmacy, but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more additional ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. Desirably, each tablet contains from about 0.5 mg to about 750 mg of the active ingredient, and each cachet or capsule contains from about 0.5 mg to about 750 mg of the active ingredient. Suitable formulations for topical application, e.g. to the skin and eyes, include aqueous solutions, suspensions, ointments, creams, gels or sprayable formulations, for example, for delivery by aerosol or the like. They are thus particularly suited for use in topical, including cosmetic, formulations well-known in the art. Such may contain solubilizers, stabilizers, tonicity enhancing agents, buffers and preservatives. Formulations suitable for topical application can be prepared e.g. as described in U.S. Pat. No. 4,784,808. Formulations for ocular administration can be prepared, e.g., as described in U.S. Pat. Nos. 4,829,088 and 4,960,799.

### 7.0. EXAMPLES

### 7.1. Example 1

### Aβ Secretion Assay

To test compounds and compositions capable of modulating Aβ levels, H4 neuroglioma cells expressing APP695NL and CHO cells stably expressing wild-type human APP751 and human mutant presenilin 1 (PS1) M146L are used. Generation and culture of these cells have been described. See Murphy et al., J. Biol. Chem., 274(17):11914-11923 (1999); Murphy et al., J. Biol. Chem., 275(34):26277-26284 (2000). To minimize toxic effects of the compositions and compounds, the H4 cells are incubated for 6 hours in the presence of the various compositions and compounds. To evaluate the potential for toxic effects of the compositions and compounds, additional aliquots of cells are incubated in parallel with each composition or compound. The supernatants are analyzed for the presence of lactate dehydrogenase (LDH) as a measure of cellular toxicity.

After incubating the cells with the compositions and compounds for a predetermined time period, sandwich enzyme-linked immunosorbent assay (ELISA) is employed to measure secreted Aβ (Aβ42 and/or Aβ40) levels as described previously. Murphy et al., J. Biol. Chem., 275(34):26277-26284 (2000). For cell culture studies serum free media samples are collected following 6-12 hours of conditioning, Complete Protease Inhibitor Cocktail added (PIC; Roche), and total Aβ concentration measured by 3160/BA27 sandwich ELISA for AP40 and 3160/BC05 sandwich ELISA for Aβ42. All measurements are performed in triplicate. Antibody 3160 is an affinity purified polyclonal antibody raised against A 1-40. HRP conjugated monoclonal antibodies BA27 for detection of Aβ40 and BC05 for detection of Aβ42 have been previously described. Suzuki et al., Science, 264(5163):1336-1340 (1994).

### 7.2. Example 2

### Determination of COX Inhibition Activity

In vitro cellular COX inhibition can be determined using specific assays for inhibition of COX-1 and COX-2 (Kalgutkar et al. J. Med Chem., 43:2860-2870 (2000)). Another art-known cellular assay for determining COX inhibition is based on the production of prostaglandin-E₂ from exogenous arachidonic acid in cells expressing COX-1, COX-2, or a combination thereof. COX enzymes (prostaglandin H synthase) catalyze the rate-limiting step in prostaglandin synthesis from arachidonic acid. Cell lines are known and available that express at least one form of the enzyme. For example, a human skin fibroblast line can be induced with IL-1 to synthesize COX-2, and a kidney epithelial cell line 293 has been stably transfected to constitutively express COX-1. In these assays, arachidonic acid can be added exogenously to increase signal to readably detectable levels. Thus, the amount of prostaglandin-E₂ in the extracellular medium can be assayed by radioimmunoassay, for measuring COX activity. IC₅₀ values for compounds for COX-1 and COX-2 can be determined by an ordinary skilled artisan. Anti-inflammatory activities of compounds can be determined using the art-known rat/mouse paw edema assay as described in Penning et al. J. Med Chem., 40:1347-1365 (1997).

For a further description of assays, cell line, and techniques capable of assessing COX inhibitory activity and Aβ₄₂ lowering activity see, e.g., WO 01/78721, and references cited therein, all of which are incorporated herein in their entirety.

### 7.3. Example 3

### Additional Alzheimer's Disease Model Assays

The levels of the Aβ peptide can be measured in conditioned medium and in lysates from cultured neuroblastoma cells transfected with an APP expression vector (Proc. Nat. Acad. Sci. USA 93:13170 (1996)). Neuronal survival and protection can be assessed with cultured neuronal cells challenged with neurotoxic factors such as the Aβ42 peptide. At various time points, cell death or viability is measured by apoptotic assay or cell counting (J. Neurobiol. 25:585, (1994); Brain Res. 706:328 (1996)). Neurite extension can be assessed with neuronal cells that are seeded in culture and the number and length of neurites that form after 16 to 20 hrs are recorded (J. Neurobiol. 25:585 (1994); J. Neurosci. 14:5461, (1994)).

### 7.4 Example 4

### Treatment of Alzheimer's Disease with R-Flurbiprofen-Statin Combination or Conjugate

R-flurbiprofen can be administered twice daily as tablets containing 400 mg of active ingredient or as a capsule containing 400 mg of the active ingredient. A higher dose can be administered to the patient in need of such treatment which can involve the patient taking e.g., a 800 mg dose of R-flurbiprofen in the morning and a 800 mg dose of R-flurbiprofen in the evening. Typically, for the treatment of mild-to-moderate Alzheimer's disease, an individual is diagnosed by a doctor as having the disease using a suitable combination of observations. One criterion indicating a likelihood of mild-to-moderate Alzheimer's disease is a score of about 15 to about 26 on the MMSE test. Another criteria indicating mild-to-moderate Alzheimer's disease is a decline in cognitive function. R-flurbiprofen can also be administered in liquid or dosage forms. The dosages can also be divided or modified, and taken with or without food. For example, the 400 mg dose can be divided into two 200 mg tablets or capsules.

Depending on the stage of the disease, the NSAID (i.e., R-flurbiprofen) can also be administered twice daily in liquid, capsule, or tablet dosage forms where the dose has various amounts of R-flurbiprofen (i.e., 850 mg, 750 mg, 700 mg, 650 mg, 600 mg, 550 mg, 500 mg, 450 mg, 350 mg, 300 mg, 250 mg, 200 mg, 150 mg, and 100 mg). Again, the dosages can also be divided or modified, and taken with or without food. The doses can be taken during treatment with other medications for treating Alzheimer's disease or symptoms thereof. For example, the NSAID can be administered twice daily as a tablet containing 400 mg of active ingredient (i.e., R-flurbiprofen) and a cholesterol lowering effective amount of a statin is administered once daily (i.e, a tablet having from about 1 mg to 100 mg atorvastatin; from about 0.5 mg to about 100 mg of simvastatin; from about 1 mg to 100 mg of lovastatin; from about 1 mg to about 100 mg of fluvastatin; from about 0.5 mg to about 50 mg pravastatin; from about 0.01 mg to about 1.5 mg cerivastatin; from about 1 mg to about 50 mg rosuvastatin; and from about 1 mg to about 100 mg of pitavastatin).

Patients having mild-to-moderate Alzheimer's disease undergoing the treatment regimen of this example with R-flurbiprofen doses of about 400 mg to 800 mg and a cholesterol lowering effective amount of a statin can experience a lessening in decline of cognitive function (as measured by the ADAS-cog or CDR sum of boxes), plaque pathology, and/or biochemical disease marker progression.

### 7.5 Example 5

### Prevention of Alzheimer's Disease with R-Flurbiprofen-Statin Conjugate or Combination

Prior to the onset of symptoms of Alzheimer's disease or just at the very beginning stages of the disease, patients desiring prophylaxis against Alzheimer's disease can be treated with a combination of R-flurbiprofen and a statin. Those needing prophylaxis can be assessed by monitoring assayable disease markers, detection of genes conferring a predisposition to the disease, other risks factors such as age, diet, other disease conditions associated with Alzheimer's disease.

The patient desiring prophylaxis against Alzheimer's disease or prophylaxis of a worsening of the symptoms of Alzheimer's disease can be treated with R-flurbiprofen and a statin in an amount sufficient to delay the onset or progression of symptoms of Alzheimer's disease. For example, a patient can be treated with 800 mg of NSAID (i.e., R-flurbiprofen) twice daily and once daily with a cholesterol lowering effective amount of a statin (i.e, a tablet having from about 1 mg to 100 mg atorvastatin; from about 0.5 mg to about 100 mg of simvastatin; from about 1 mg to 100 mg of lovastatin; from about 1 mg to about 100 mg of fluvastatin; from about 0.5 mg to about 50 mg pravastatin; from about 0.01 mg to about 1.5 mg cerivastatin; from about 1 mg to about 50 mg rosuvastatin; or from about 1 mg to about 100 mg of pitavastatin). Alternatively, the statin and R—NSAID can be formulated in a single tablet for administration once or twice daily. Another preventive regimen involves administering to the patient 400 mg of R-flurbiprofen twice daily and twice daily with a cholesterol lowering effective amount of a statin (i.e, a total daily dosage of from about 1 mg to 100 mg atorvastatin; from about 0.5 mg to about 100 mg of simvastatin; from about 1 mg to 100 mg of lovastatin; from about 1 mg to about 100 mg of fluvastatin; from about 0.5 mg to about 50 mg pravastatin; from about 0.01 mg to about 1.5 mg cerivastatin; from about 1 mg to about 50 mg rosuvastatin; or from about 1 mg to about 100 mg of pitavastatin). The amounts of these active ingredients can be modified to lessen side-effects and/or produce the most therapeutic benefit. For example, 200 mg of R-flurbiprofen twice daily can be administered to reduce sides-effects associated with the use of higher levels of the active ingredient. The preventive treatment can also be, e.g., treatment on alternating days with R-flurbiprofen, or alternating weeks. Lastly the NSAID statin conjugates described herein can be administered to the patient desiring (or needing) prophylaxis against Alzheimer's disease. The amount of conjugate needed is that which produces substantially the same effect as when the therapeutic agents (R—NSAID and statin) are administered in a non-conjugated form.

### 7.6 Example 6

### Co-Formulation of R-Flurbiprofen with Statins

### R-Flurbiprofen Atorvastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Atorvastatin | 10 mg |

### R-Flurbiprofen Simvastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Simvastatin | 20 mg |

### R-Flurbiprofen Lovastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Lovastatin | 40 mg |

### R-Flurbiprofen Fluvastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Fluvastatin | 80 mg |

### R-Flurbiprofen Pravastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Pravastatin | 40 mg |

### R-Flurbiprofen Cerivastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Cerivastatin | 0.4 mg |

### R-Flurbiprofen Rosuvastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Rosuvastatin | 10 mg |

### R-Flurbiprofen Pitavastatin Tablets

| Ingredient | Amount |
|---|---|
| R-Flurbiprofen | 400 mg |
| Microcrystalline Cellulose | 392 mg |
| Colloidal Silicon Dioxide | 4 mg |
| Magnesium Stearate | 4 mg |
| Pitavastatin | 10 mg |

The tablets are prepared using art known procedures and the amounts ingredients listed above can be modified to obtain an improved formulation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1. A pharmaceutical composition comprising R-flurbiprofen or a pharmaceutically acceptable salt or ester thereof and an HMG-CoA reductase inhibitor or a pharmaceutically acceptable salt or ester thereof, and a pharmaceutically acceptable carrier.

2. The composition of claim 1, comprising about 400 mg R-flurbiprofen or an equivalent amount of a pharmaceutically acceptable salt thereof.

3. The composition of claim 2, wherein said composition is substantially free of S-flurbiprofen.

4. The composition of claim 1, comprising about 800 mg R-flurbiprofen or an equivalent amount of a pharmaceutically acceptable salt thereof.

5. The composition of claim 4, wherein said composition is substantially free of S-flurbiprofen.

6. The composition of claim 1 wherein said HMG-CoA reductase inhibitor is a statin.

7. The composition of claim 1 wherein said HMG-CoA reductase inhibitor is pravastatin or pitavastatin.

8. The composition of claim 1, comprising an amount of said R-flurbiprofen or pharmaceutically acceptable salt or ester thereof when administered in a single dose to a fasting individual sufficient to produce a plasma Cₘₐₓ of about 25-150 μg per mL per dose and an AUC (area under curve of concentration versus time; total drug exposure) of from about 200 hr·μg/mL to about 600 hr·μg/mL.

9. A method of treating or delaying the onset of Alzheimer's disease in an individual comprising treating the individual with an effective amount of R-flurbiprofen and an HMG-CoA reductase inhibitor or ezetimibe, sufficient to treat or delay the onset of Alzheimer's disease.

10. The method of claim 9, wherein said individual is diagnosed of mild cognitive impairment.

11. The method of claim 9, wherein said HMG-CoA reductase inhibitor is a statin.

12. The method of claim 9, comprising administering to the individual a composition comprising an effective amount of R-flurbiprofen or a pharmaceutically acceptable salt or ester thereof and a statin or ezetimibe, or a pharmaceutically acceptable salt or ester thereof.

13. The method of claim 12 said statin is pravastatin or pitavastatin.

14. The method of claim 12, wherein said composition comprises about 400 mg R-flurbiprofen or an equivalent amount of a pharmaceutically acceptable salt thereof.

15. The method of claim 14, wherein said composition is substantially free of S-flurbiprofen.

16. The method of claim 12, wherein said composition comprises about 800 mg R-flurbiprofen or an equivalent amount of a pharmaceutically acceptable salt thereof.

17. The method of claim 16, wherein said composition is substantially free of S-flurbiprofen.

18. The method of claim 12, wherein said composition comprises an amount of said R-flurbiprofen or pharmaceutically acceptable salt or ester thereof when administered in a single dose to a fasting individual sufficient to produce a plasma Cₘₐₓ of about 25-150 μg per mL per dose and an AUC (area under curve of concentration versus time; total drug exposure) of from about 200 hr·μg/mL to about 600 hr·μg/mL.

19. A method of treating or delaying the onset of Alzheimer's disease in an individual comprising administering to said individual an effective amount of R-flurbiprofen to provide in said individual an AUC₁₂ (area under curve of concentration in a 12-hour window, i.e., total drug exposure in a 12-hour window) of from about 200 hr·μg/mL to about 450 hr·μg/mL.

20. A conjugate comprising an NSAID or a pharmaceutically acceptable salt or ester thereof and an HMG-CoA reductase inhibitor or a pharmaceutically acceptable salt or ester thereof.

21. The conjugate of claim 23 where the NSAID is an R—NSAID.

22. The conjugate of claim 24 where the R—NSAID is R-flurbiprofen.

23. A composition comprising a combination of R-flurbiprofen or a pharmaceutically acceptable salt or ester thereof and ezetimibe or a pharmaceutically acceptable salt or ester thereof.
